(19) United States
(12) Patent Application Publication
(10) Pub. No.: US 2015/0050722 A1
(43) Pub. Date: Feb. 19, 2015

(54) Cell Culture Strain Array Systems and Methods for Using the Same

(71) Applicants: CS Laboratory Technologies LLC, Mountain View, CA (US); The Board of Trustees of the Leland Stanford Junior University, Palo Alto, CA (US)

(72) Inventors: Chelsey S. Simmons, Gainesville, FL (US); Beth L. Pruitt, Stanford, CA (US); Joo Yong Sim, Stanford, CA (US); Philipp R. Bächtold, Wallisellen (CH)

(21) Appl. No.: 14/376,739

(22) PCT Filed: Feb. 5, 2013
(86) PCT No.: PCT/US2013/024774

§ 371 Date: Aug. 5, 2014

Related U.S. Application Data
(60) Provisional application No. 61/595,590, filed on Feb. 6, 2012.

(51) Int. Cl. C12M 1/42; C12M 1/32; C12M 1/00

(52) U.S. Cl. CPC C12M 35/04; C12M 23/26; C12M 23/12
USPC 435/288.4; 435/289.1; 435/305.2

(57) ABSTRACT

Aspects of the invention include cell culture systems that include a cell culture plate operatively coupled to a plenum device. The plenum device includes a base component, one or more wall components configured to define a bounded volume, and one or more strain platens that are configured to support the cell culture plate when the cell culture plate is operatively coupled to the plenum device. In some instances, the plenum device includes a pressure modulator configured to provide a substantially uniform pressure in the bounded volume upon application of an external pressure source via an internal side opening in a wall component. Aspects of the invention further include system components and kits thereof, as well as methods of using the systems, e.g., in cell culture applications, which may include, e.g., candidate agent screening applications.

### CROSS REFERENCE TO RELATED APPLICATIONS

Under 35 U.S.C. §119(e), this application claims priority to the filing date of U.S. Provisional Patent Application Ser. No. 61/595,590, filed on Feb. 6, 2012, the disclosure of which application is herein incorporated by reference in its entirety.

### STATEMENT OF GOVERNMENT SPONSORED SUPPORT

This invention was made with Government support under contract R21 HL089027 awarded by National Institutes of Health (NIH), contract CBE 0735551 awarded by the National Science Foundation (NSF), and contract RC1-00151-1 awarded by the California Institute of Regenerative Medicine (CIRM). The Government has certain rights in this invention.

### BACKGROUND

In vivo, cells undergo deformation and stress in normal physiology. Skin cells, for example, resist high mechanical stress, and simple monolayers of epithelia and endothelia are regularly subjected to bending and shear forces. Cells are also subjected to high strains, e.g., as the heart pumps, lungs expand, and biceps flex. To study these processes in vitro, commercially available systems have been developed that can apply both tensile and compressive strains to large cell populations, and numerous custom systems have been reported. Using these systems, strain fields have been applied to a variety of biological models, including muscle, stem, bone and endothelial cells. Tensile strain has been shown to affect numerous biological processes, such as, e.g., cell alignment, morphology, differentiation, proliferation, apoptosis and signaling.

However, most systems only apply a single strain profile across an entire plate or population; thus many prior studies have been limited to relatively few, significantly different strain levels, and have therefore failed to allow comparisons across experiments. Inconsistencies in the strain fields of commercial devices have also been implicated in contradictory results. As a result, microfluidics and soft lithography techniques have been used to apply strain to small populations of cells, using innovative geometries to create controlled variations in strain or porous substrates for biomimetic functions. Other approaches have used microscale devices to mechanically strain a single cell at a time. While silicon-based microdevices are easily and reliably calibrated, the manipulation of cells and devices by hand is time consuming and therefore only facilitates low throughput studies.

To create higher throughput systems, some investigators hydraulically pressurize and lift an array of cylindrical posts to biaxially stretch polymer membranes. A major drawback of this system, however, is that the posts rise to different heights, and therefore place samples at different focal planes, complicating imaging processes while stretching. Other investigators have created devices that address the focal plane issue, but such devices are limited to only one strain level. Furthermore, multi-strain devices often circulate, or pool media among samples experiencing different strain levels, and such pooling couples the different cell populations through paracrine signaling pathways and eliminates the possibility to investigate secreted factors and small molecule signaling under strain.

### SUMMARY

Aspects of the invention include cell culture systems that include a cell culture plate operatively coupled to a plenum device. The plenum device includes a base component, one or more wall components configured to define a bounded volume, and one or more strain platens that are configured to support the cell culture plate when the cell culture plate is operatively coupled to the plenum device. In some instances, the plenum device includes a pressure modulator configured to provide a substantially uniform pressure in the bounded volume upon application of an external pressure source via an internal side opening in a wall component. Aspects of the invention further include system components and kits thereof, as well as methods of using the systems, e.g., in cell culture applications, which may include, e.g., candidate agent screening applications.

In some embodiments, aspects of the cell culture system include a plenum device, the plenum device including a base component, a wall component configured to define a bounded volume having a bottom that is a surface of the base component, and a pressure modulator configured to provide a substantially uniform pressure inside the bounded volume upon application of an external pressure source via an internal side opening in the wall component.

In some embodiments, the pressure modulator includes one or more structures extending from at least one of the surface of the base component and an inner surface of the wall component. In some embodiments, the pressure modulator includes a plurality of strain platens extending from at least one of the surface of the base component and an inner surface of the wall component. In some embodiments, the structures are uniformly spaced, while in some embodiments, the structures are non-uniformly spaced.

In some embodiments, a device is configured to impart a mechanical strain on a flexible membrane material that forms a pliant bottom of a well of a cell culture plate operatively coupled thereto. In some embodiments, the strain is a substantially isotropic mechanical strain, while in some embodiments the strain is a substantially anisotropic mechanical strain. In some embodiments, the device is configured to impart a mechanical strain gradient on two or more wells of a cell culture plate operatively coupled thereto.

In some embodiments, the device comprises two or more strain platens configured to impart different mechanical strains on different wells of a cell culture plate operatively coupled thereto. In some embodiments, the two or more strain platens have different cross-sectional shapes. In some embodiments, the two or more strain platens have different cross-sectional dimensions.

In some embodiments, the base component and the wall component of the device are integrated into a single unit, while in some embodiments the base component and the wall component are separable from one another. In some embodiments, the wall component comprises a single internal side opening, while in some embodiments, the wall component comprises two or more internal side openings. In some embodiments, the bounded volume of the plenum device has a volume ranging from about 10 to about 120 cubic centimeters.

Aspects of the invention include a cell culture system that includes a plenum device that includes a base component, a wall component configured to define a bounded volume having a bottom that is a surface of the base component, and a pressure modulator configured to provide a substantially uniform pressure in the bounded volume upon application of an external pressure source via an internal side opening in the wall component, and a cell culture plate that includes two or more cell culture wells, each well having a pliant bottom.

In some embodiments, the pressure modulator includes one or more structures extending from at least one of the surface of the base component and an inner surface of the wall component. In some embodiments, the pressure modulator includes a plurality of strain platens extending from at least one of the surface of the base and an inner surface of the wall component. In some embodiments, the structures are uniformly spaced, while in some embodiments the structures are non-uniformly spaced.

In some embodiments, the device is configured to impart a mechanical strain on the pliant bottoms of the wells of the cell culture plate. In some embodiments, the strain is a substantially isotropic mechanical strain, while in some embodiments the strain is a substantially anisotropic mechanical strain. In some embodiments, the plenum device is configured to impart a mechanical strain gradient on the pliant bottoms of two or more wells of the cell culture plate. In some embodiments, the device comprises two or more strain platens configured to impart different mechanical strains on the pliant bottoms of two or more wells of the cell culture plate. In some embodiments, the two or more strain platens have different cross-sectional shapes. In some embodiments, the two or more strain platens have different cross-sectional dimensions.

In some embodiments, the plenum device is configured to maintain at least a portion of the pliant bottoms of the wells of the cell culture plate in substantially the same focal plane when a mechanical strain is imparted to the pliant bottoms. In some embodiments, the system includes a control system configured to modulate the pressure inside the bounded volume of the plenum device. In some embodiments, the control system is a pneumatic control device. In some embodiments, the control system is a hydraulic control device. In some embodiments, the control system is a closed-loop control system. In some embodiments, the control system is configured to modulate the pressure in the bounded volume of the plenum device according to a waveform. In some embodiments, the control system includes a microprocessor. In some embodiments, the microprocessor includes a program that, when executed, causes the control system to modulate the pressure in the bounded volume of the plenum device. In some embodiments, the program is configured to accept a user input. In some embodiments, the program is configured to display a graphical user interface.

In some embodiments, the system includes a fluid transport system operatively coupled to the cell culture plate. In some embodiments, the fluid transport system is configured to deliver one or more fluids to one or more wells of the cell culture plate. In some embodiments, the fluid transport system is configured to withdraw a quantity of fluid from one or more wells of the cell culture plate. In some embodiments, the fluid transport system comprises a fluid reservoir. In some embodiments, the cell culture plate is operatively coupled to a cell culture incubator. In some embodiments, the incubator is configured to modulate and/or control at least one of the temperature and the gaseous environment of the wells of the cell culture plate.

In some embodiments, the system includes a pressure source. In some embodiments, the system includes an imaging device. In some embodiments, the cell culture plate includes a lid that is configured to allow retrieval of the contents of one or more wells of the cell culture plate. In some embodiments, the system includes a stimulation device configured to deliver an electrical stimulation to the contents of one or more wells of the cell culture plate. In some embodiments, the stimulation device is operatively coupled to a well of the cell culture plate. In some embodiments, the cell culture plate includes an electrode array. In some embodiments, the electrode array includes electrodes operatively coupled to two or more wells of the cell culture plate.

In some embodiments, the cell culture plate includes a composite structure that is configured to mechanically stabilize the cell culture plate. In some embodiments, the cell culture plate is configured to promote protein attachment to the wells of the cell culture plate. In some embodiments, the cell culture plate is configured to promote binding of a non-biological material to the cell culture substrate of the cell culture plate. In some embodiments, the cell culture plate includes a secondary cell culture surface.

Aspects of the invention include a method of culturing cells that involves placing a cell in a cell culture plate of a cell culture system that includes a plenum device that includes a base component, a wall component configured to define a bounded volume having a bottom that is a surface of the base component, and a pressure modulator configured to provide a substantially uniform pressure in the bounded volume upon application of an external pressure source via an internal side opening in the wall component, and a cell culture plate comprising two or more cell culture wells, each having a pliant bottom, and applying a pressure to the bounded volume of the plenum device to impart a mechanical strain to the pliant bottoms of the wells of the cell culture plate, thereby imparting a mechanical strain to the cell.

In some embodiments, the method involves imparting a substantially isotropic mechanical strain to the pliant bottoms. In some embodiments, the method involves imparting a substantially anisotropic mechanical strain to the pliant bottoms. In some embodiments, the plenum device is configured to impart a mechanical strain gradient to the pliant bottoms of the cell culture plate. In some embodiments, the cell is a stem cell. In some embodiments, the cell is attached to a tissue culture scaffold.

Aspects of the invention include a method of evaluating the activity of a candidate agent, the method involving contacting a cell with a candidate agent, wherein the cell is present in a cell culture system that includes a plenum device that includes a base component, a wall component configured to define a bounded volume having a bottom that is a surface of the base component, and a pressure modulator configured to provide a substantially uniform pressure in the bounded volume upon application of an external pressure source via an internal side opening in the wall component, and a cell culture plate that includes two or more cell culture wells each having a pliant bottom, modulating the pressure in the bounded volume of the plenum device to induce a mechanical strain in the pliant bottoms, thereby imparting a mechanical strain to the cell, and assaying the cell and/or the cell culture medium to evaluate the activity of the candidate agent.

In some embodiments, the method involves imparting a substantially isotropic mechanical strain to the pliant bottoms. In some embodiments, the method involves imparting a substantially anisotropic mechanical strain to the pliant bottoms. In some embodiments, the method involves imparting a mechanical strain gradient to the pliant bottoms. In some embodiments, the cell is attached to a tissue culture scaffold. In some embodiments, the cell is a stem cell.

In some embodiments, the candidate agent is evaluated for cellular differentiation activity, gene expression modulatory activity, protein production modulatory activity, or signaling pathway modulatory activity. In some embodiments, the method is a high throughput method.

Aspects of the invention includes kits that include a plenum device that includes a base component, a wall component configured to define a bounded volume having a bottom that is a surface of the base component, and a pressure modulator configured to provide a substantially uniform pressure in the bounded volume upon application of an external pressure source via an internal side opening in the wall component, and a cell culture plate that includes two or more cell culture wells, each having a pliant bottom.

### BRIEF DESCRIPTION OF THE DRAWINGS

FIG. 1 is an illustration of a cell culture plate according to one embodiment of the present invention.

FIG. 2 is an illustration of a plenum device according to one embodiment of the present invention.

FIG. 3 is a cutaway illustration of a plenum device according to one embodiment of the present invention.

FIG. 4 is an illustration of a plenum device according to one embodiment of the present invention.

FIG. 5 is an illustration of a plenum device according to one embodiment of the present invention.

FIG. 6 is a cutaway illustration of a plenum device according to one embodiment of the present invention.

FIG. 7 is a top-view illustration of a plenum device according to one embodiment of the present invention.

FIG. 8 is an illustration of one side of a plenum device according to one embodiment of the present invention.

FIG. 9 is an illustration of a cell culture plate aligned with a plenum device according to one embodiment of the present invention.

FIG. 10 is a cutaway illustration of a cell culture plate aligned with a plenum device according to one embodiment of the present invention.

FIG. 11 is an illustration of a cell culture system according to one embodiment of the present invention, including a cell culture plate, a plenum device, a control system, and a computer processor.

FIG. 12 is a schematic illustration of a control system according to one embodiment of the present invention operatively coupled to a plenum device.

FIG. 13 is an illustration showing a top view and a side view of cells being cultured in the wells of a cell culture plate that is operatively coupled to a plenum device and a control system according to one embodiment of the present invention. In Panel A, the vacuum source is turned off, and the pliant bottom of the cell culture well has not been stretched over the strain platen. In Panel B, two different strain platens having a circular cross-sectional shape are depicted. The first strain platen has a larger cross-sectional diameter, which results in a lower amount of strain when the pliant bottom of the cell culture well is stretched over the strain platen. The second strain platen has a smaller cross-sectional diameter, which results in a higher amount of strain when the pliant bottom of the cell culture well is stretched over the strain platen. Panel B depicts the deformation of the pliant bottoms of the cell culture wells over the strain platens when the vacuum source has been turned on. The arrows in Panel B indicate the direction in which a force is applied due to the change in pressure inside the bounded volume of the plenum device.

FIG. 14 is an illustration showing a top view of cells being cultured under two different strain conditions. In Panel A, the cell culture well is aligned with a strain platen that has a circular cross-sectional shape, and the cells are subject to equibiaxial/isotropic strain when the pliant bottom of the cell culture well is stretched over the strain platen. In Panel B, the cell culture well is aligned with a strain platen that has an oval cross-sectional shape, and the cells are subjected to uniaxial/anisotropic strain when the pliant bottom of the cell culture well is stretched over the strain platen.

FIG. 15 is an illustration showing a top view and a side view of cells being cultured on a secondary cell culture surface in a well of a cell culture plate that is operatively coupled to a plenum device according to one embodiment of the present invention. In Panel A, an attached vacuum source is turned off, and the pliant bottom of the cell culture well is not stretched over the strain platen. Panel B depicts the deformation of the pliant bottom of the cell culture wells over the strain platen when the vacuum source has been turned on. The arrows in Panel B indicate the direction in which a force is applied due to the change in pressure inside the bounded volume of the plenum device. When the vacuum source is turned on, the pliant bottom of the cell culture well is stretched over the strain platen, and the secondary cell culture surface and attached cells experience mechanical strain.

FIG. 16, Panel A is an illustration of a finite element model (FEM) of the amount of strain that is induced in the pliant bottom of a cell culture well at various distances from the center of the well. Panel B is a graph showing directional strains as a function of distance from the center of the well for a 2.0 mm diameter circular strain platen. Panel C is a graph showing strain as a function of distance from the center of the well for circular strain platens having the indicated diameter.

FIG. 17, Panels A and B are graphs showing measured strains as a function of strain platen diameter for four different strain platens.

FIG. 18 is a graph showing measured strains at various times of operation for four different strain platens.

FIG. 19 is a microscope image of cells that were cultured in a cell culture plate and subjected to mechanical strain. The amount of strain experienced by the cells is represented by the difference in area between the two depicted triangles that are superimposed over the microscope image.

FIG. 20 shows bright field and fluorescent microscope images taken from cells that were cultured in cell culture plates and subjected to different amounts of mechanical strain for various amounts of time. Nuclei and F-actin within the cells were stained and characterized to determine the effect of the applied mechanical strain.

### DETAILED DESCRIPTION

Aspects of the invention include cell culture systems that include a cell culture plate operatively coupled to a plenum device. The plenum device includes a base component, one or more wall components configured to define a bounded volume, and one or more strain platens that are configured to support the cell culture plate when the cell culture plate is operatively coupled to the plenum device. In some instances, the plenum device further includes a pressure modulator configured to provide a substantially uniform pressure in the bounded volume upon application of an external pressure source via an internal side opening in a wall component. Aspects of the invention further include system components and kits thereof, as well as methods of using the systems, e.g., in cell culture applications, which may include, e.g., candidate agent screening applications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of embodiments of the invention in greater detail, aspects of the systems and devices of various embodiments are reviewed first in greater detail, followed by a discussion of methods and kits according to certain embodiments of the invention.

### Systems and Devices

Aspects of the invention include systems and devices configured for applying mechanical strain to a population of cells. In certain embodiments, the system includes a cell culture plate, a plenum device, and a control system. Each of these components is now further described in greater detail.

### Cell Culture Plate

As summarized above, aspects of the invention include a cell culture plate that is configured to be operatively coupled to a plenum device. The cell culture plate generally includes a plurality of sides (or walls) and a plurality of individual wells in which cells can be cultured. In some embodiments, the cell culture plate also includes a lid. The bottom surface of the wells generally comprises a flexible membrane material, or pliant bottom, that is configured to be stretched in response to an external force.

Cell culture plates in accordance with embodiments of the invention may have wells that are spaced according to well-known industry standards, such as, e.g., those described by the Society for Laboratory Automation and Screening standards (SLAS). As such, the spacing of the wells of a cell culture plate in accordance with embodiments of the invention can match standard 96-well, 48-well, 24-well, 12-well and/or 6-well spacing formats that are generally used for in vitro cell culture. In other embodiments, the spacing of the wells can deviate from industry-standard spacing formats, and can be, for example, irregularly and/or regularly spaced according to a customized design. In some embodiments, a cell culture plate may contain a different number of cell culture wells than are present on an industry-standard cell culture plate. For instance, in some embodiments, a cell culture plate may include, e.g., up to about 25 wells, up to about 50 well, up to about 75 wells, up to about 100 wells, such as up to about 150 wells, such as up to about 200 wells, such as up to about 250 wells, such as up to about 300 wells, such as up to about 350 wells, such as up to about 400 wells, such as up to about 450 wells, such as up to about 500 wells or more.

Cell culture plates in accordance with embodiments of the invention may have wells that have a variety of different cross-sectional shapes, including but not limited to circular, oblong, square, or rectangular shapes. By “cross-sectional shape of a cell culture well” is meant the shape that would be seen by an observer looking directly down at the well from above the cell culture plate. In some embodiments, all of the wells of a cell culture plate may have the same cross-sectional shape, e.g., all of the wells may be circular. In some embodiments, a cell culture plate may include two or more wells that have different cross-sectional shapes. For example, in some embodiments, a cell culture plate may include a plurality of wells that have a circular cross-sectional shape as well as a plurality of wells that have a square cross-sectional shape. In addition, in some embodiments, the shape or geometry of the bottom surface of the wells can also vary. For example, cell culture plates in accordance with embodiments of the invention may have round bottom wells and/or flat bottom wells.

The depth of the wells of a cell culture plate in accordance with embodiments of the invention is generally configured to accommodate a suitable amount of a cell culture medium to be used when culturing cells therein. In some embodiments, the depth of the wells is such that the wells are able to hold (or contain) a certain volume of cell culture medium per unit area of the base of the well. For example, in some embodiments, the depth of the wells is such that the wells can hold about 0.10 mL, up to about 0.15 mL, up to about 0.20 mL, up to about 0.25 mL, up to about 0.30 mL, up to about 0.40 mL, up to about 0.50 mL, up to about 0.60 mL, up to about 0.70 mL, up to about 0.75 mL or more of cell culture medium per square centimeter of the base of the well.

Cell culture plates in accordance with embodiments of the invention may also have a variety of different shapes and sizes. For example, in some embodiments, cell culture plates may be square, wherein the cell culture plate has four sides, each side having the same length. In some embodiments, the cell culture plate may be rectangular. In some embodiments, cell culture plates may be circular, oblong or any other suitable shape.

Cell culture plates in accordance with embodiments of the present invention may also include a combination of different cell culture well sizes and spacing formats within the same cell culture plate. For example, in some embodiments, two or more standard cell culture plate layouts may be combined to form a large cell culture plate that can be used for high throughput experiments. For example, in some embodiments, a 2×2 grid of standard 96-well plates may be combined to create a large cell culture plate that includes 384 wells. In some embodiments, a standard 96-well plate may be combined with a standard 12-well plate. Any of a variety of suitable combinations of well sizes, shapes, and spacing formats may be used to create cell culture plates in accordance with embodiments of the invention.

Cell culture plates in accordance with some embodiments of the invention may have a footprint that is similar to or the same as other industry-standard cell culture plates, such as the SLAS standard cell culture plates referenced above. By “footprint of a cell culture plate” is meant the area that is covered by the cell culture plate when the cell culture plate is placed on a surface. In some embodiments, a cell culture plate may have a footprint that matches other industry-standard cell culture plates to facilitate analysis of the cells grown in the cell culture plate using standard equipment, such as, e.g., microscopes, plate readers, assay equipment, cell culture fluid handling equipment, and the like.

In some embodiments, cell culture plates may include markings and/or other design elements that may be used to designate a plate orientation or otherwise identify one or more of the wells on the plate. Such features may include, but are not limited to, e.g., a distinctive corner that is different from the other corners of the cell culture plate and can be used to correctly orient the plate for analysis or handling. Examples of markings include standard row and column indicators, such as columns marked with numbers and rows marked with letters, or vice versa, to facilitate identification of each individual cell culture well on the plate. In some embodiments, row and/or column labels may be imprinted on a cell culture plate using, e.g., printed dyes, encapsulated dyes, embossing, and/or raised lettering and/or numbering. Markings may also be applied to a cell culture plate using such standard techniques as writing with a pen or pencil, etching, scratching, or otherwise marking the cell culture plate.

Cell culture plates in accordance with embodiments of the invention generally include a flexible membrane material that forms a pliant bottom of the cell culture wells. The flexible membrane material can be stretched in order to apply a desired amount of mechanical strain to the cells being cultured. The thickness of the flexible membrane material is only limited by the ability of the material to be stretched to the desired extent.

In some embodiments, the flexible membrane material has a thickness ranging from about 10 μm, up to about 50 μm, up to about 100 μm, up to about 150 μm, up to about 200 μm, up to about 250 μm, up to about 300 μm, up to about 350 μm, up to about 400 μm, up to about 450 μm, up to about 500 μm, up to about 550 μm, up to about 600 μm, up to about 650 μm, up to about 700 μm, up to about 750 μm, up to about 800 μm, up to about 850 μm, up to about 900 μm, up to about 950 μm, up to about 1 mm.

In some embodiments, the material that forms the walls of the wells of the cell culture plate has a thickness that is substantially greater than the thickness of the flexible membrane material that forms the base of the wells so that the flexible membrane material will stretch preferentially. For example, in certain embodiments, the walls of the wells of the cell culture plate are made from a material that is greater than or equal to five times the thickness of the flexible membrane material that forms the pliant bottoms of the wells.

Cell culture plates in accordance with embodiments of the invention may generally include a lid that is configured to maintain sterility and protect the contents of the cell culture wells from particulates and/or other contaminants while still allowing gas transport, such as, e.g., the transport of oxygen and carbon dioxide, to occur. In some embodiments, the lid of the cell culture plate may be removable, while in some embodiments the lid may be permanently bonded to the cell culture plate. In some embodiments, the lid may be reversibly bonded to the cell culture plate and/or may be locked in place, such that the lid remains in place until the locking mechanism is opened, released, removed or otherwise separated from the cell culture plate.

Cell culture plates in accordance with embodiments of the invention may include a fluid transport system that is configured to deliver fluid to the wells of the cell culture plate and/or to remove fluid from the wells of the cell culture plate while maintaining the sterility of the culture. For example, in some embodiments, the cell culture plate includes a fluid transport system comprising a series of channels that are configured to deliver, e.g., cell culture medium, soluble factors, and/or agents of interest to the wells of the cell culture plate. In some embodiments, the channels of the fluid transport system may have a diameter ranging in size from about 5 μm or less up to about 25 μm, up to about 50 μm, up to about 100 μm, up to about 250 μm, up to about 500 μm, up to about 750 μm, up to about 1 mm, up to about 2 mm, up to about 3 mm, up to about 4 mm, or up to about 5 mm or more.

In some embodiments, the fluid transport system may be completely located in the lid of the cell culture plate, while in other embodiments the fluid transport system may be completely located in the wall portions surrounding the cell culture wells. In some embodiments, the fluid transport system may be completely located in the base of the cell culture plate or completely located in the flexible membrane material. In some embodiments, the fluid transport system may have elements that are located in different portions of the cell culture plate. For example, in certain embodiments, components or portions of the fluid transport system may be located in the lid of the cell culture plate, as well as in the base of the cell culture plate, as well as in the flexible membrane material, as well as in the wall portions of the cell culture plate.

In certain embodiments, the fluid transport system may additionally include valve components, pumps, reservoirs and/or other fluid control components that may be used to control the flow of fluid through the fluid transport system. In some embodiments, the fluid transport system may have one or more connecting components that are configured to connect to an external fluid handling system that can be used to deliver fluid to the fluid transport system. For example, in some embodiments, the fluid transport system may include one or more connecting components that are configured to fluidly connect a source of, e.g., cell culture medium with the fluid transport system. In such embodiments, after connecting the source of cell culture medium to the fluid transport system, a specified amount of the cell culture medium may be transported to one or more designated wells of the cell culture plate.

In some embodiments, the fluid transport system may be configured to collect fluid from the wells of the cell culture plate. For example, in such embodiments, a specified amount of fluid may be transported from one or more wells of the cell culture plate and transferred to another specified location. For instance, in certain embodiments, the fluid transport system can transfer a specified amount of fluid from one well into another well. In certain embodiments, the fluid transport system can transfer a specified amount of fluid from a well into a designated collection component, such as, e.g., a syringe, a fluid collection reservoir, or a sample tube. Fluid collected from the wells by the fluid transport system may include supernatant alone, or may include both supernatant and cells, such as, e.g., detached cells, such as trypsinized cells.

In some embodiments, the fluid transport system is configured for dynamic application of fluid shear stress to the contents of the wells of the cell culture plate. For example, in certain embodiments, the fluid transport system is configured to apply a specified amount of fluid shear stress to cells that are cultured in the cell culture plate. The magnitude, frequency and duration of the applied fluid shear stress can be modulated using standard control system that controls the movement of fluid through the fluid transport system. In certain embodiments, an automated syringe pump can be used to move fluid through the fluid transport system to apply the desired amount of shear stress to the cells. In certain embodiments, the fluid transport system is configured to continuously perfuse the cell culture wells with a specified amount of fluid, e.g., a cell culture medium.

In some embodiments, the fluid transport system may be configured to interact with other instruments. For example, in some embodiments, the fluid transport system may be configured to interact with an apparatus, such as an assay device, wherein a fluid connection is established between the assay device and the fluid transport system of the cell culture plate, and a designated amount of fluid is withdrawn from one or more wells of the cell culture plate and used to perform an assay. In some embodiments, the fluid transport system may be configured to interact with a control system, wherein a user can specify an amount of fluid to be transferred to or from a given well of the cell culture plate, and the control system will carry out the instruction.

Cell culture plates in accordance with embodiments of the invention may include a stimulation device configured to apply an external stimulation to the contents of the wells of the cell culture plate. In some embodiments, the stimulation device components may be located within one or more wells of the cell culture plate, such as on the sides of the wells or on the base of the wells, or may be located on the lid of the cell culture plate. In some embodiments, the stimulation device components may be located on the outside of the cell culture plate, such as, for example, where a stimulation device and/or components thereof is located on the top of the lid of the cell culture plate, or is located on the underside of the base of one or more of the wells of the cell culture plate. In some embodiments, the stimulation device and/or its components may be detachable, wherein the stimulation device components may be attached to the cell culture plate and used for a specified period of time, and then may be removed from the cell culture plate without otherwise interrupting or interfering with the culturing of cells in the wells of the plate. In some embodiments, the stimulation device and/or its components may be permanently attached to the cell culture plate. For example, in some embodiments, the stimulation device components may be embedded within or permanently attached to the material of the wells of the cell culture plate, the base of the cell culture plate, or the lid of the cell culture plate.

In some embodiments, a stimulation device may be an electrical stimulation device that is configured to apply an electrical stimulation to the contents of one or more wells of the cell culture plate. For example, in some embodiments, the stimulation device may apply an electric voltage of constant or variable strength to the contents of a well. In some embodiments, the stimulation device may apply an electric current of constant or variable strength to the contents of a well. In some embodiments, a stimulation device may be configured to apply electromagnetic radiation of a specified wavelength to the contents of a well. For example, in some embodiments, the stimulation device may apply light, e.g., from a light-emitting diode, to a well. In some embodiments, the stimulation device may apply laser light to the contents of a well. The duration, magnitude and frequency of the application of the stimulation may be controlled by, e.g., standard control systems and/or devices that are configured to modulate the activity of the stimulation device.

Cell culture plates in accordance with embodiments of the invention may include a detection device configured to collect data from one or more of the wells of the cell culture plate. In some embodiments, the detection device and/or its components may be located within one or more wells of the cell culture plate, such as on the sides of the wells or on the base of the wells, or may be located on the lid of the cell culture plate. In some embodiments, the detection device and/or its components may be located on the outside of the cell culture plate, such as, for example, where a detection device is located on the top of the lid of the cell culture plate, or is located on the underside of the base of one or more of the wells of the cell culture plate. In some embodiments, the detection device components may be detachable, wherein the detection device components may be attached to the cell culture plate and used for a specified period of time, and then may be removed from the cell culture plate without otherwise interrupting or interfering with the culturing of cells in the wells of the plate. In some embodiments, the detection device components may be permanently attached to the cell culture plate. For example, in some embodiments, the detection device and/or its components may be embedded within or permanently attached to the material of the wells of the cell culture plate, the base of the cell culture plate, or the lid of the cell culture plate.

In some embodiments, a detection device may detect an optical characteristic of the contents of a well of the cell culture plate. For example, in some embodiments, a detection device may detect, e.g., the optical density of the cell culture that is growing within a well of the cell culture plate. In some embodiments, a stimulation device and a detection device may be configured to interact with one another to collect data from the contents of a well of the cell culture plate. For example, in certain embodiments, the stimulation device may be configured to apply a stimulus to the contents of one or more wells of the cell culture plate, and the detection device may be configured to measure a specified characteristic of the contents of the wells in response to the stimulus.

Cell culture plates in accordance with embodiments of the invention can be made from any of a variety of suitable materials, including but not limited to polymers (e.g., tissue culture plastic materials) metals, or ceramics. Suitable materials generally include water-insoluble, fluid-impervious, sterile or sterilizable, typically thermoplastic materials that are substantially chemically non-reactive with the fluids and other materials typically used in cell culture applications. Suitable materials include, but are not limited to, e.g., polystyrene or polyvinyl chloride with or without copolymers, polyethylenes, polystyrene-acrylonitrile, polypropylene, polyvinylidine chloride, and the like. In some embodiments, cell culture plates may comprise glass. In some embodiments, cell culture plates may comprise a silicone material, such as, e.g., polydimethylsiloxane (PDMS). In some embodiments, a cell culture plate may comprise a material that has previously been approved by the FDA and/or has desirable biocompatibility characteristics, e.g., is a biocompatible material.

In some embodiments, a cell culture plate is made entirely from the same material. Suitable manufacturing techniques for making such cell culture plates include, but are not limited to, e.g., injection molding. In such embodiments, the flexible membrane material that forms the pliant bottoms of the cell culture wells is made from the same material as the remainder of the cell culture plate, and the cell culture plate is manufactured with the flexible membrane material attached to the remainder of the cell culture plate. In some embodiments, various components of a cell culture plate are fabricated separately, and the cell culture plate is then assembled from the separate parts. For example, in some embodiments, the flexible membrane material is a separate component that is bonded to, fastened to, adhered to, disposed upon, or otherwise connected to the remainder of the cell culture plate such that the flexible membrane material forms the bottom portion of one or more wells of the cell culture plate.

In some embodiments, the cell culture plate is a composite that is made from components and/or different materials that have different properties. For example, in some embodiments, a portion or component of the cell culture plate can be made from a material that has different mechanical properties, e.g., is substantially-rigid or semi-rigid as compared to the other materials used to make the cell culture plate. In certain embodiments, the cell culture plate comprises one or more reinforcing components that are made from a rigid material, and/or are configured to mechanically reinforce the wells and/or the walls of the cell culture plate. In some embodiments, the reinforcing components may be removable, such that the reinforcing components can be repeatedly removed or inserted as needed to provide mechanical support to the cell culture plate.

In some embodiments, a portion or component of the cell culture plate can be made from an electrically conductive material and/or may comprise an electrically conductive region. For example, in some embodiments, a composite cell culture plate may comprise a component that is made from an electrically conductive material, such as, e.g., gold, platinum, or copper. In certain embodiments, the electrically conductive material component may be an encapsulated insert, and/or may be removable such that it can be removed or inserted as desired. In some embodiments, the electrically conductive material may be functionalized with one or more surface components of interest, such as, e.g., a coating of a protein, such as avidin or streptavidin.

In certain embodiments, a cell culture plate can include a recess, or channel, filled with a conductive fluid, such as, e.g., ionized water or an indium alloy. In some embodiments, a cell culture plate can include one or more void spaces or channels intended for fluid flow and/or fluid collection.

Cell culture plates in accordance with embodiments of the present invention generally include a flexible membrane material that forms the bottom surface of the cell culture wells and can be stretched to apply a mechanical strain to the contents of the wells. In some embodiments, the flexible membrane material comprises a material that is configured to stretch to a specified degree and yet still maintain certain desired optical characteristics. For example, in some embodiments, the flexible membrane material comprises a material that is substantially optically transparent both in its stretched and un-stretched states so that cells being cultured in the wells of the plate can be continuously imaged using, e.g., standard microscopy and/or imaging equipment. For example, in some embodiments, the flexible membrane material comprises polydimethylsiloxane (PDMS).

Cell culture plates in accordance with embodiments of the invention may include various surface treatments on the cell culture substrates to facilitate desired interactions between the cells and the substrate. By “cell culture substrates” is meant the surfaces, including the bottom and/or the interior sides of the wells, of the cell culture plate upon which cells are grown. For example, in some embodiments, a cell culture substrate may be patterned with physical surface features, including but not limited to, e.g., a ring, a divot, or an indentation, to constrain cell attachment to a specified location. In some embodiments, a cell culture substrate may be patterned with physical surface features such as, e.g., one or more ridges, trenches, or indentations in a desired pattern to facilitate alignment, grouping, or controlled interactions between the cells that are cultured on the substrate. In some embodiments, a cell culture substrate may be patterned with other materials, such as, e.g., one or more conductive materials, such as, e.g., gold, aluminum, carbon nanotubes, and/or carbon nanoparticles to form electrically-active regions of the cell culture substrate. In some embodiments, a cell culture substrate may be contacted with one or more proteins or molecules that promote attachment of cells and/or other molecules of interest to the cell culture substrate. Examples of such proteins include, but are not limited to, e.g., avidin and streptavidin. In some embodiments, a cell culture substrate can be contacted with various chemicals to control and/or regulate the attachment of cells to the cell culture substrate. Examples of chemicals that can be used for surface treatments include but are not limited to, e.g., silanes, polyethylene glycol, acrylates, and the like. Any of the surface treatments disclosed herein may be used either separately or in combination with one another.

In some embodiments, a secondary cell culture surface may be placed in one or more wells of the cell culture plate. By “secondary cell culture surface” is meant a component or material that is separate from the cell culture plate, and which is placed into the wells of the cell culture plate to serve as a surface upon which cells can grow, or to serve as a matrix within which cells can grow. In some embodiments, a cell culture substrate can be treated to permanently or reversibly attach a secondary cell culture surface to the cell culture plate. For example, in some embodiments, a secondary cell culture surface may be, e.g., a hydrogel, such as, e.g., polyacrylamide, polyethylene glycol, collagen, gelatin, or suitable combinations thereof, such as, e.g., Matrigel™, that is placed into the wells of the cell culture plate. Suitable secondary cell culture surfaces may also comprise, e.g., proteins (e.g., growth factors, signaling factors) small molecules, large molecules, and/or any other suitable agents of interest. Cells can be grown on or within the secondary cell culture surface. In some embodiments, a secondary cell culture surface can be reversibly attached to the cell culture plate with the use of temperature-sensitive chemicals such as, e.g., poly-N-isopropylacrylamide.

In some embodiments, isolated tissue segments may be placed inside the wells of the cell culture plate and cultured therein. In certain such embodiments, a secondary cell culture surface may also be placed in the well, such that the isolated tissue segment can be cultured within, or upon the secondary cell culture surface. In some embodiments, a tissue segment may be either reversibly or permanently attached to a cell culture substrate or a secondary cell culture surface. Attachment of such isolated tissue segments can be accomplished by, e.g., treating a cell culture substrate and/or a secondary cell culture surface with, e.g., silane and glutaraldehyde or benzophenone. In some embodiments, isolated tissue segments may be placed in, on, within, or adjacent to a secondary cell culture surface and either permanently or reversibly attached thereto.

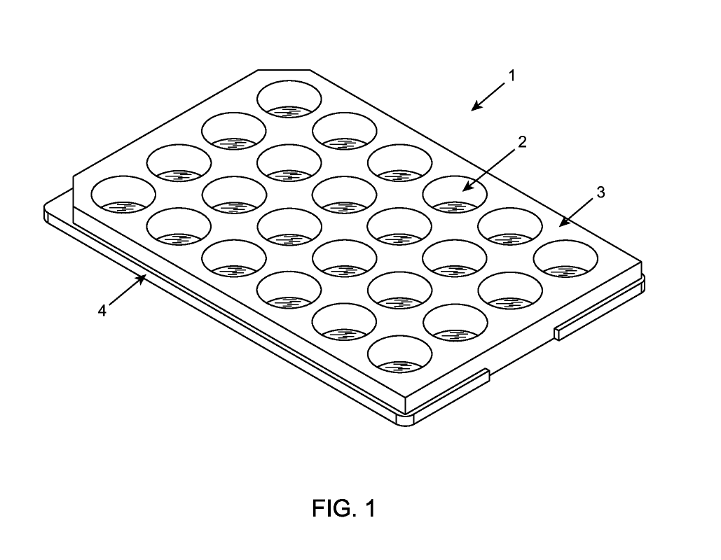

Referring now to FIG. 1, an embodiment of a cell culture plate according to the present invention is depicted. The depicted cell culture plate 1 is rectangular in shape and has 24 cell culture wells 2. The wall portion of the cell culture plate 3 is made entirely from a single material. The flexible membrane material, or pliant bottom 4 is adhered to the wall portion of the cell culture plate to form the bottoms of the cell culture wells 2.

### Plenum Device

As summarized above, aspects of the invention include a plenum device that is configured to support a cell culture plate, as described above. The plenum device generally includes a base, one or more wall components configured to define a bounded volume, wherein the bottom of the bounded volume is defined by a surface of the base of the plenum device, and a plurality of strain platens. In some embodiments, the base and the one or more wall components may be formed as one unit (i.e., formed from the same piece of material), while in some embodiments, the one or more wall components may be separable from the base of the plenum device. In some embodiments, when a cell culture plate is operatively coupled to the plenum device, one or more of the strain platens are aligned with one or more wells of the cell culture plate, and the cell culture plate is supported, or suspended, at least partially, by one or more of the strain platens. In some embodiments, the plenum device includes one or more support components, such as, e.g., support ledges and/or support pillars that provide support to the cell culture plate when the cell culture plate is operatively coupled to the plenum device.

In some embodiments, one or more of the strain platens extend from the base of the plenum device in the form of a pillar, or column. In some embodiments, one or more of the strain platens extend from one or more of the wall components of the plenum device. In some embodiments, strain platens may extend from both the base of the plenum device as well as from one or more of the wall components of the plenum device.

Plenum devices in accordance with embodiments of the invention may have strain platens that are spaced such that when a cell culture plate, as described above, is operatively coupled to the plenum device, the strain platens are aligned with one or more of the wells of the cell culture plate. In some embodiments, the strain platens are uniformly spaced, while in other embodiments, the strain platens are non-uniformly spaced. In certain embodiments, a plenum device may include both uniformly and non-uniformly spaced strain platens. Any of a variety of suitable alignment and spacing strategies may be employed such that the strain platens are located in desired positions on the plenum device.

Strain platens in accordance with embodiments of the invention may have a variety of different shapes and geometries. For example, in some embodiments, strain platens may have different cross-sectional shapes in order to impart different types of strain on the flexible membrane material of the cell culture plate when the flexible membrane material is stretched over the strain platen. By “cross-sectional shape of a strain platen” is meant the shape that would be seen by an observer looking directly down at the strain platen from above. In some embodiments, strain platens may have circular, oblong, square or rectangular cross-sectional shapes. In some embodiments, all of the strain platens in a plenum device may have the same cross-sectional shape, while in some embodiments two or more strain platens in a plenum device may have different cross-sectional shapes. In certain embodiments, a plenum device may comprise an array of strain platens that have a number of different cross-sectional shapes.

Strain platens in accordance with embodiments of the invention may vary in height and may have a variety of different dimensions. In general, strain platens in accordance with embodiments of the invention are configured to extend from their origination point on the plenum device to a suitable position so as to make contact with the pliant bottoms of the wells of a cell culture plate that is operatively coupled to the plenum device. Depending on the geometry of the cell culture plate that is operatively coupled to the plenum device, the strain platens may be taller or shorter than the wall component of the plenum device in order to make sufficient contact with the pliant bottoms of the cell culture wells.

In some embodiments, the height of the strain platens is substantially the same as the height of the wall component of the plenum device. In some embodiments, the strain platens may be taller than the wall component of the plenum device, such that the strain platens extend above the top, or upper surface of the wall component of the plenum device. In some embodiments, the strain platens may be shorter than the wall component of the plenum device, such that the top of the strain platens is below the top, or upper surface of the wall component of the plenum device.

In certain embodiments, the height of the strain platens may be, e.g., 100 μm or more, such as 200 μm or more, such as 300 μm or more, such as 400 μm or more, such as 500 μm or more, such as 750 μm or more, such as 1 mm or more, such as 2 mm or more, such as 3 mm or more, such as 4 mm or more, such as 5 mm or more, such as 6 mm or more, such as 7 mm or more, such as 8 mm or more, such as 9 mm or more, such as 10 mm or more, such as 20 mm or more.

The cross-sectional dimensions of the strain platens can vary widely. By “cross-sectional dimension of a strain platen” is meant the width, thickness, or diameter of the strain platen. For example, in some embodiments, strain platens may have the same cross-sectional shape but may have different cross-sectional dimensions in order to impart different magnitudes of strain on the flexible membrane material of the cell culture plate when the flexible membrane material is stretched over the strain platen. In some embodiments, all of the strain platens in a plenum device may have the same cross-sectional dimensions. In some embodiments, two or more different strain platens in a plenum device may have different cross-sectional dimensions. In certain embodiments, a plenum device may comprise an array of strain platens that have a number of different cross-sectional dimensions. In some embodiments, a plenum device may comprise two or more strain platens that have different cross-sectional shapes, as well as two or more strain platens that have different cross-sectional dimensions. In certain embodiments, a plenum device may comprise a plurality of strain platens having an array of different cross-sectional shapes as well as an array of different cross-sectional dimensions. Any of a variety of suitable combinations of strain platen cross-sectional shapes and strain platen cross-sectional dimensions may be included in plenum devices in accordance with embodiments of the invention.

As described above, some embodiments of the plenum device may include one or more support components in addition to the strain platens. Support components in accordance with embodiments of the invention may generally have any cross-sectional shape and may have a variety of heights and cross-sectional dimensions as needed to provide support to the cell culture plate when the cell culture plate is operatively coupled to the plenum device. In general, support components in accordance with embodiments of the invention are long enough to extend from the base of the plenum device up to the surface of the plenum device that is contacted by a cell culture plate when the cell culture plate is operatively coupled to the plenum device. In some embodiments, support components may be ledges that provide support to the cell culture plate along its periphery. In some embodiments, support components may be pillars or columns that extend from the base of the plenum device. In some embodiments, when the cell culture plate is operatively coupled to the plenum device, the support components are not aligned with the wells of the cell culture plate. Instead, the support components contact the cell culture plate in locations other than the area occupied by the bottom portion of the wells.

Strain platens and/or support components in accordance with embodiments of the invention may be solid or hollow. In some embodiments, a plenum device may include both hollow and solid strain platens and/or support components. In some embodiments, one or more strain platens and/or support components are hollow, and various components may be placed inside. For example, in certain embodiments, a hollow strain platen or support component may contain, e.g., imaging components, stimulation components, and/or detection components. In some embodiments, strain platens and/or support components may be thin-walled and may therefore be transparent or semi-transparent, allowing imaging to take place through at least a portion thereof. In some embodiments, hollow strain platens and/or support components may have one or more openings that allow access to the internal portion thereof. In such embodiments, one or more of the openings may be covered with a suitable material, e.g., a suitable transparent or non-transparent material. In certain embodiments, one or more of the openings in a hollow strain platen and/or support component may be covered with, e.g., transparent glass. In other embodiments, the one or more openings may be left uncovered.

In some embodiments, the support components and/or the strain platens may have rounded or smoothed edges so as not to tear or otherwise damage the flexible membrane material of the cell culture plate. In certain embodiments, a plenum device may comprise support features that are configured to support a standard SLAS cell culture plate, as described above.

Plenum devices in accordance with embodiments of the invention may include a pressure modulator that comprises one or more support components and/or strain platens that are arranged in a pattern and configured to uniformly distribute pressure within the bounded volume of the plenum device when a pressure source (e.g., a vacuum source) is operatively connected to the plenum device. For example, in some embodiments, a pressure modulator may comprise a plurality of support components that are spaced in a pattern throughout the plenum device in order to uniformly (i.e., evenly) distribute pressure within the bounded volume of the plenum device when a cell culture plate is operatively coupled to the plenum device and a pressure source is connected to the plenum device. In some embodiments, a pressure modulator may comprise a plurality of strain platens that are spaced in a pattern throughout the plenum device in order to evenly distribute pressure within the bounded volume of the plenum device when a cell culture plate is operatively coupled to the plenum device and a pressure source is connected to the plenum device. In certain embodiments, a pressure modulator may comprise a combination of strain platens and support components that are spaced in a pattern throughout the plenum device in order to evenly distribute pressure within the bounded volume of the plenum device when a cell culture plate is operatively coupled to the plenum device and a pressure source is connected to the plenum device.

In some embodiments, the spacing between a plurality of support components and/or a plurality of strain platens may be selected such that the pressure within the bounded volume of the plenum device is evenly distributed when a pressure source is applied to the bounded volume via, e.g., an internal side opening in a wall component of the plenum device. In some embodiments, the support components and/or strain platens that make up the pressure modulator may be evenly spaced with respect to each other, while in some embodiments, the support components and/or strain platens that make up the pressure modulator may be unevenly spaced with respect to each other, such that some of the support components and/or strain platens are located very close together, while other support components and/or strain platens are located further from one another. For example, in some embodiments, a pressure modulator may comprise, e.g., a plurality of support components that are located around the periphery of the bounded volume and are closely spaced, as well as a plurality of strain platens and additional support components that are located in a central area of the bounded volume and are spaced further from one another. By uniformly distributing the pressure within the bounded volume of the plenum device, the support components and/or strain platens provide for even, or uniform, application of pressure to the pliant bottom of each well of the cell culture plate that is aligned with a strain platen.

In some embodiments, two structures that make up a portion of pressure modulator (e.g., two support components, two strain platens, or one support component and one strain platen) may be spaced such that the distance between the structures ranges from about 100 μm, up to about 250 μm, up to about 500 μm, up to about 750 μm, up to about 1 mm, up to about 2 mm, up to about 3 mm, up to about 4 mm, up to about 5 mm, up to about 6 mm, up to about 7 mm, up to about 8 mm, up to about 9 mm, up to about 1 cm, up to about 2 cm, up to about 3 cm, up to about 4 cm, up to about 5 cm or more.

Plenum devices in accordance with embodiments of the invention also generally include one or more access channels that have at least one internal opening in one or more wall portions of the plenum device to allow a fluid to access the bounded volume. The access channels have at least one external opening that may be located on the top, the bottom, and/or the side of the plenum device. In some embodiments, an access channel has a single internal opening and a single external opening. In such embodiments, the access channel may pass straight through the wall portion of the plenum device, or may include a plurality of turns before exiting the plenum device at the external opening. In some embodiments, the access channel includes one or more turns within the wall portion and/or the base portion of the plenum device. In certain embodiments, an access channel may include one internal opening and may include two or more external openings. In other embodiments, an access channel may include two or more internal openings, and only one external opening. In some embodiments, an access channel may have two or more internal openings, as well as two or more external openings. In some embodiments, an access channel will have an appropriate number of Y-junctions, wherein the access channel is divided from one channel into two channels. In embodiments having two or more internal openings, such openings may be located on the same portion of the plenum device, e.g., on the same wall portion of the plenum device, or may be located on different portions of the plenum device, e.g., on different wall portions. In embodiments having two or more external openings, such openings may be located on the same portion of the plenum device, e.g., on the same wall portion of the plenum device, or may be located on different portions of the plenum device, e.g., on different wall portions, on the top surface of the plenum device, or on the bottom surface of the plenum device. Access channels in accordance with embodiments of the invention may comprise a network of separate or connected channels that pass throughout the plenum device.

In general, a fluid control component is operatively connected to an access channel at the one or more external openings of the access channel. For example, in certain embodiments, a vacuum source may be connected to the plenum device at an external opening of the access channel via, e.g., connective tubing. In some embodiments, the plenum device may have two or more access channels, or may have an access channel that has two or more external openings. In such embodiments, two or more of the external openings of the access channel may be connected to the same fluid control component. For example, two or more external openings may be connected to, e.g., the same vacuum source, by connective tubing that forms a Y-junction.

Plenum devices in accordance with embodiments of the invention may include alignment and/or attachment features to facilitate operatively coupling a cell culture plate to the plenum device. For example, in some embodiments, a plenum device may include alignment marks on its surface that instruct proper placement or alignment of a cell culture plate. Such alignment marks may include, e.g., arrows, lines, ridges, grooves, and the like. In some embodiments, a plenum device may include one or more surface features, such as, e.g., divots, holes, ridges, grooves, and/or tabs, that mate or align with corresponding structures or features on a cell culture plate so as to ensure proper placement or alignment of the cell culture plate on the plenum device.

Plenum devices in accordance with embodiments of the invention can be made from a variety of different materials, including but not limited to polymers, metals and/or ceramics. In some embodiments, a plenum device is made from a single type of material, while in other embodiments a plenum device may be a composite that is made from two or more different types of materials. In general, a plenum device can be made from any rigid or semi-rigid material. In certain embodiments, a plenum device can be made from a sterilizable material. In certain embodiments, a plenum device is made from one or more materials that are resistant to degradation and/or deformation when exposed to sterilization agents or solvents, such as, e.g., ethanol or isopropanol, steam sterilization procedures, such as, e.g., autoclaving, and/or gas sterilization procedures, such as, e.g., exposure to ethylene oxide gas, oxygen plasma, or ozone. Examples of suitable materials include but are not limited to polyethylene, polystyrene, polytetrafluoroethylene (a.k.a. PTFE, Teflon™), polyoxymethylene (a.k.a. acetal, Delrin™), acrylic, glass, metal, ceramic, or combinations thereof.

Plenum devices in accordance with embodiments of the invention can be manufactured using a variety of suitable techniques, including but not limited to machining, molding, injection molding, etching, lithography, laser ablation, laser etching and/or combinations thereof. In some embodiments, a plenum device can be made from one single part, e.g., a single block or piece of material, while in other embodiments a plenum device can be made from two or more different components that are assembled to form the final plenum device.

Plenum devices in accordance with embodiments of the invention may also include various additional components, such as, e.g., connection components that are configured to connect a plenum device to a fluid source. Such connection components may be configured for reversible or permanent connection. Examples of reversible connection components include, e.g., luer lock components, quick disconnect fittings, click together or push together tubing components, and the like. In some embodiments, additional components of the plenum device may be sterilizable and/or may resist deformation or disconnection when exposed to an operating fluid under positive and/or negative pressure. For example, tubing connection components may be configured to stay connected when exposed to a vacuum or pressurized gas. Example materials that can be used include but are not limited to polyurethane and/or polyvinyl chloride.

In some embodiments, connection components, such as, e.g., tubing components, may be configured to connect two or more plenum devices in a daisy chain format, such that the two or more plenum devices share the same operating fluid source and/or are operated by the same control system. For example, in some embodiments, two plenum devices may be connected to one another via connective tubing such that a single vacuum source, controlled by a single control system, is applied to both of the plenum devices.

In some embodiments, a lubricant may be applied to the cell culture plate and/or a surface of the plenum device, such as, e.g., a surface of one or more of the strain platens, to facilitate movement of the flexible membrane material of the cell culture plate over the strain platens. In certain embodiments, a lubricant and/or a sealant may be applied to the cell culture plate and/or a surface of the plenum device to seal the cell culture plate to the plenum device such that the operating fluid is confined to the bounded volume and cannot escape past the cell culture plate. For example, in some embodiments, a sealant may be applied to the edges of the cell culture plate to form an air-tight seal between the cell culture plate and the plenum device. Suitable lubricants and/or sealing materials include but are not limited to, e.g., water and/or glycerin-based solutions or compounds. Lubricants used in some embodiments may also have specific optical properties that facilitate, e.g., imaging of cells that are present in the wells of the cell culture plate, such as, e.g., microscope immersion oil.

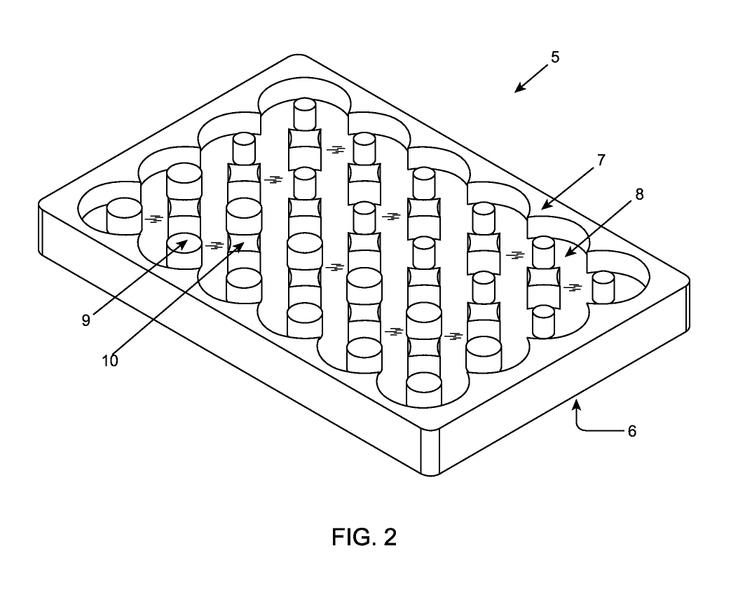

Referring now to FIG. 2, an embodiment of a plenum device according to the present invention is depicted. The depicted plenum device 5 is rectangular and has a base 6, a wall component 7, and a bounded volume 8. The plenum device has a plurality of strain platens 9 and a plurality of support components 10. The depicted strain platens 9 all have a circular cross-sectional shape, but some of the strain platens have different cross-sectional dimensions, i.e., two or more of the strain platens have a different diameter. In the depicted embodiment, the plurality of strain platens 9 and support components 10 make up a pressure modulator that evenly distributes pressure within the bounded volume 8.

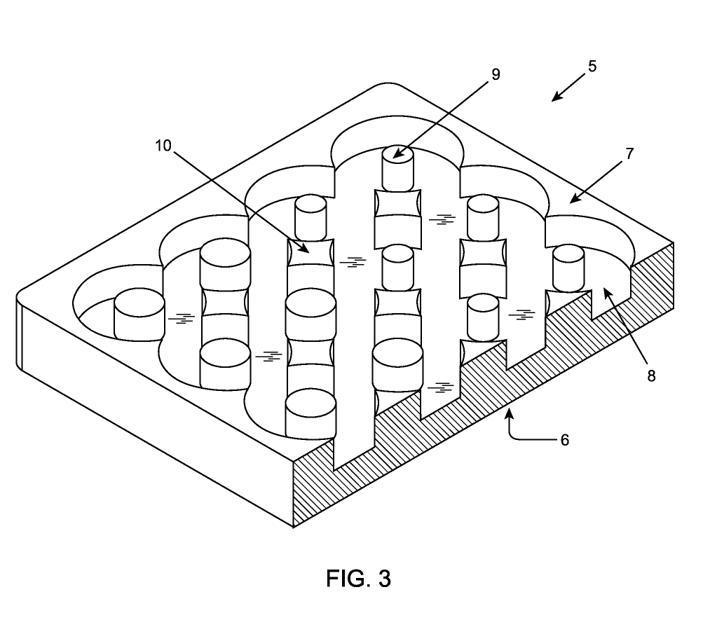

Referring now to FIG. 3, an embodiment of a plenum device according to the present invention is depicted. In this cutaway view of the plenum device 5, the base 6, wall component 7, bounded volume 8, plurality of strain platens 9 and plurality of support components 10 can be seen.

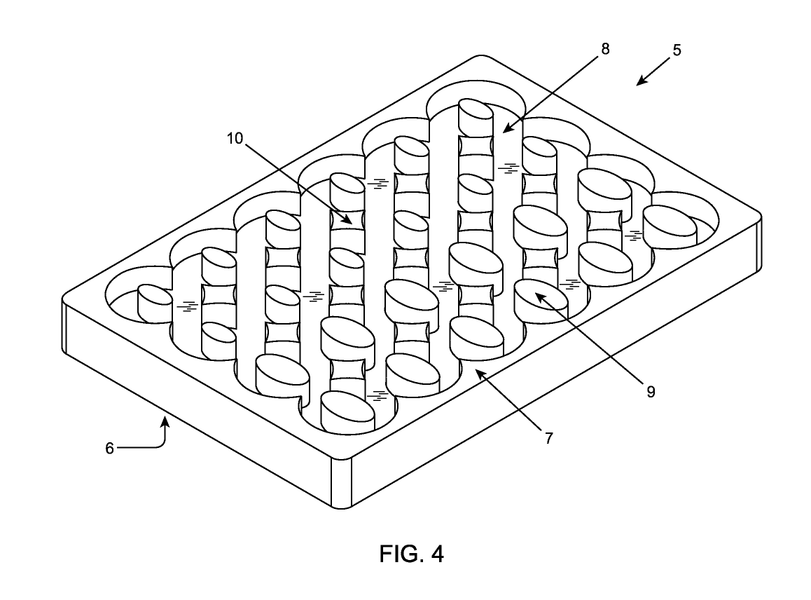

Referring now to FIG. 4, an embodiment of a plenum device according to the present invention is depicted. The depicted plenum device 5 has a base 6, a wall component 7, a bounded volume 8, a plurality of strain platens 9 and a plurality of support components 10. In the depicted embodiment, all of the strain platens 9 have an oval cross-sectional shape, but two or more of the strain platens 9 have different cross-sectional dimensions. In the depicted embodiment, the plurality of strain platens 9 and support components 10 make up a pressure modulator that evenly distributes pressure within the bounded volume 8.

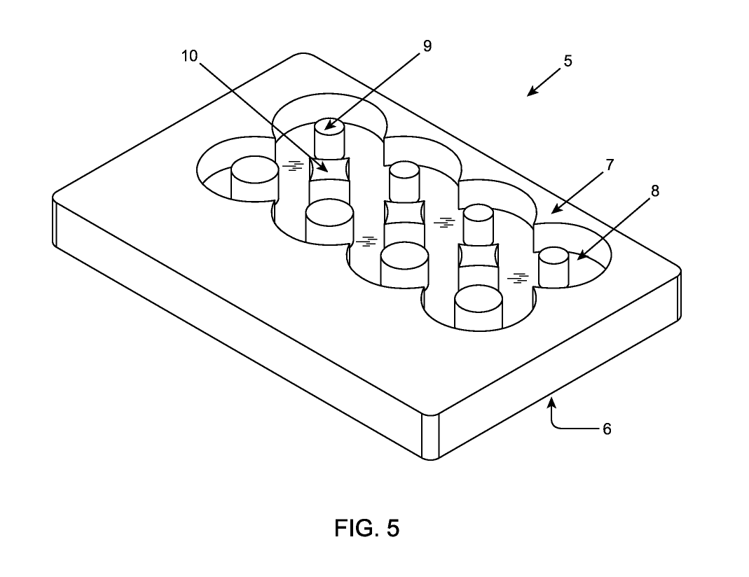

Referring now to FIG. 5, an embodiment of a plenum device according to the present invention is depicted. The depicted plenum device 5 has a base 6, a wall component 7, a bounded volume 8, a plurality of strain platens 9 and a plurality of support components 10. In the depicted embodiment, all of the strain platens 9 have the same cross-sectional shape, but two or more of the strain platens 9 have different cross-sectional dimensions. In the depicted embodiment, the wall component of the plenum device is thicker along one side. When a cell culture plate is operatively coupled to the depicted plenum device, one or more of the cell culture wells will be aligned directly over this thicker portion of the wall component. Accordingly, the pliant bottoms of these wells will not come into contact with (i.e., will not be aligned with) a strain platen and therefore will not be stretched when the pressure inside the bounded volume is modulated. These wells can therefore serve as control wells that do not experience mechanical strain.

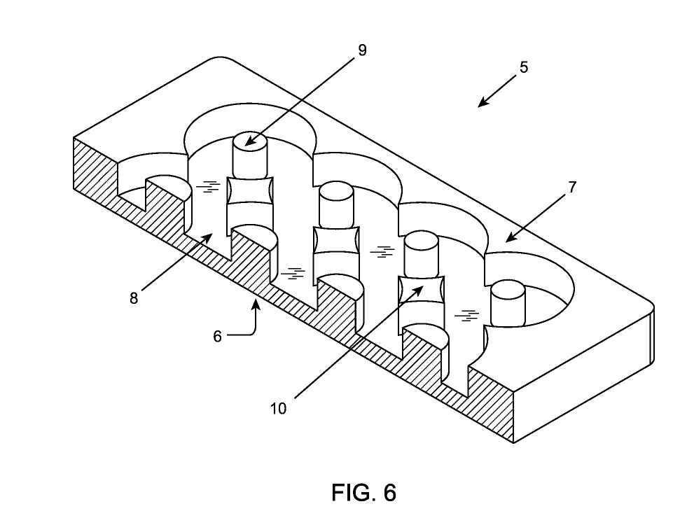

Referring now to FIG. 6, an embodiment of a plenum device according to the present invention is depicted. In this cutaway view of the plenum device 5, the base 6, wall component 7, bounded volume 8, plurality of strain platens 9 and a plurality of support components 10 can be seen. The depicted strain platens 9 have the same cross-sectional shape (i.e., a circular cross-sectional shape) but two or more of the strain platens have different cross-sectional dimensions (i.e., different diameters).

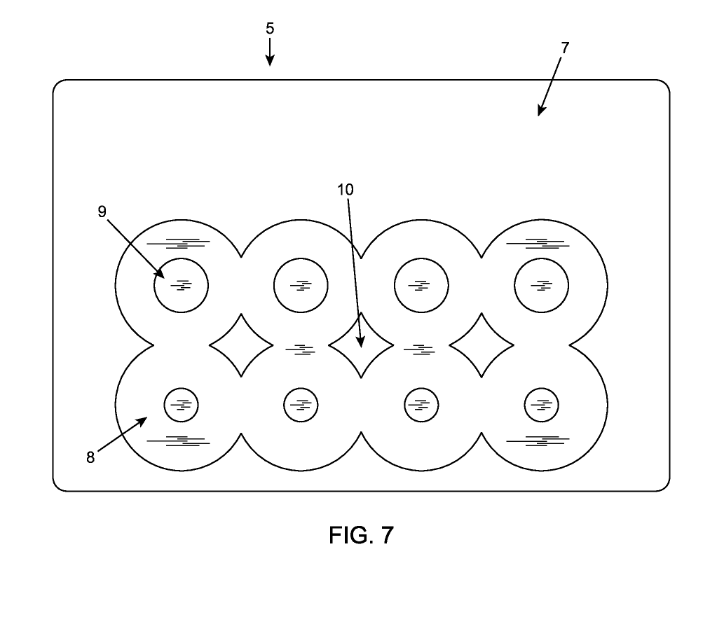

Referring now to FIG. 7, an embodiment of a plenum device according to the present invention is depicted. In this top view, the bounded volume 8, plurality of strain platens 9, and plurality of support components 10 can be seen.

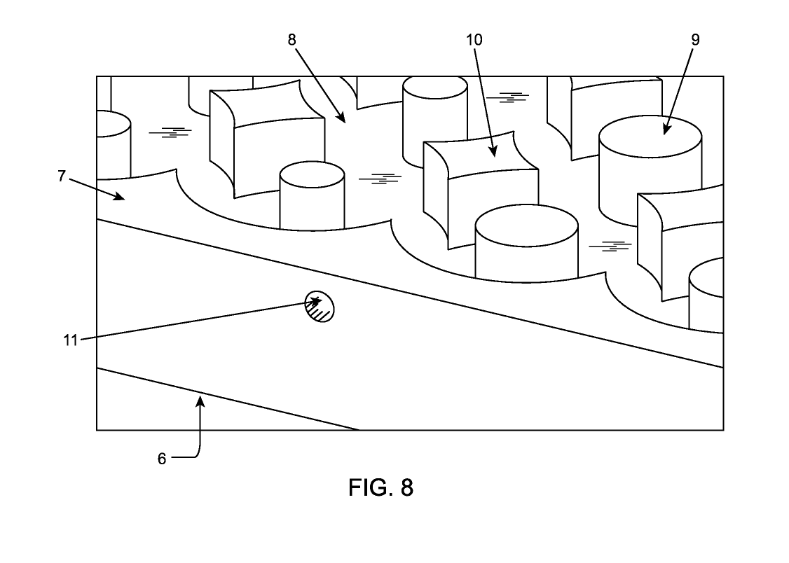

Referring now to FIG. 8, an embodiment of a plenum device according to the present invention is depicted. In the depicted embodiment, the external opening of an access channel 11 can be seen in the wall component 7. In the depicted embodiment, the plurality of strain platens 9 and support components 10 make up a pressure modulator that evenly distributes pressure within the bounded volume 8.

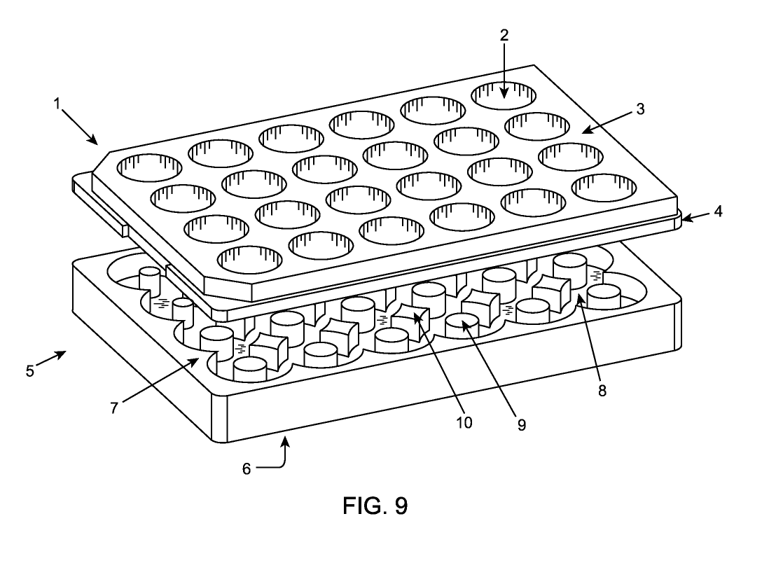

Referring now to FIG. 9, an embodiment of a cell culture plate 1 and a plenum device 5 are depicted. In the depicted embodiment, the cell culture plate is aligned with the plenum device such that the wells 2 of the cell culture plate are aligned with the strain platens 9 of the plenum device. The depicted plenum device includes a plurality of support components 10 that make contact with the cell culture plate in locations other than the bottoms of the wells of the cell culture plate. When the depicted cell culture plate 1 is operatively coupled to the depicted plenum device 5, the flexible membrane material 4 seals against the plenum device to enclose the bounded volume 8.

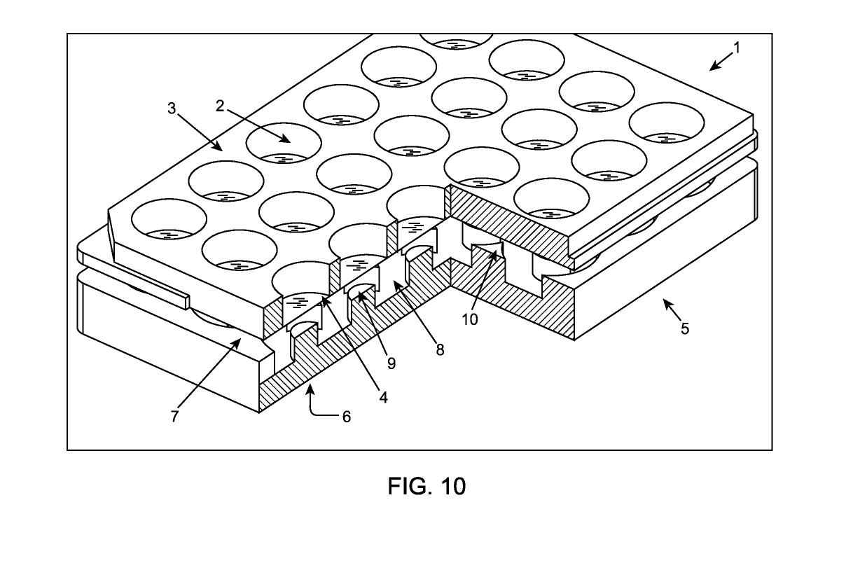

Referring now to FIG. 10, an embodiment of a cell culture plate 1 and a plenum device 5 according to the present invention are depicted. In the depicted cutaway view, the cell culture plate 1 is aligned with the plenum device 5 such that the wells 2 of the cell culture plate are aligned with the strain platens 9 of the plenum device. The support components 10 make contact with the cell culture plate in locations other than the bottoms of the cell culture wells 2. The depicted cutaway view also shows the pliant bottom 4 of the cell culture plate.

### Additional Components

In addition to the devices and components described above, aspects of the invention may also include peripheral components such as, e.g., components that are configured for the handling and maintenance of cell culture systems. For example, any of a variety of suitable cell culture incubation systems that are configured to maintain one or more desired temperature, humidity, and/or gas concentration settings or ranges (such as, e.g, a desired carbon dioxide concentration and/or a desired oxygen concentration) may be employed with the cell culture devices and systems described herein. Examples of additional peripheral components include, but are not limited to, incubators, fluid handling systems, such as, e.g., fluid systems that can be used to add and/or remove cell culture medium, supernatant, and/or cells to or from a cell culture plate, assay systems that are configured to perform various assays on the contents of a cell culture plate, or imaging systems that are configured to image the contents of a cell culture plate.

In certain embodiments, aspects of the invention may include imaging equipment and/or devices, such as, e.g., microscopes and digital image capturing equipment. For example, in some embodiments, aspects of the invention may include a microscope imaging system configured to, e.g., automatically capture images of cells that are cultured in the cell culture plate on a periodic basis. The frequency of imaging can be specified by a user, as desired.

### Control Systems

As summarized above, aspects of the invention include a control system that is configured to modulate the pressure inside the bounded area of the plenum device, described above. Control systems in accordance with embodiments of the invention can operate using a variety of different fluids, including but not limited to air, nitrogen gas, liquid water, or liquid glycerin. In some embodiments, the control system modulates the pressure within the bounded area of the plenum device by moving a fluid within the bounded area of the plenum device. In some embodiments, the control system can include a pressure source, such as a negative pressure source, e.g., a vacuum, or a positive pressure source, such as, e.g., compressed dry air. Examples of negative pressure sources include, but are not limited to, external vacuum pumps or plumbed vacuum lines. Examples of positive pressure sources include, but are not limited to, compressed gas sources, such as, e.g., a tank of compressed nitrogen gas or a plumbed dry air line.

In some embodiments, the control system may include a pressure regulator that can be used to control, or modulate the pressure between the plenum device and the pressure source. For example, in some embodiments, a control system may include a pressure regulator that reduces the pressure from a tank of compressed nitrogen gas to a specified pressure for use with the cell culture system. In some embodiments, the control system may also include other liquid-based control components, such as, e.g., pumps, valves, reservoirs, and the like to control, modulate, and/or manipulate the operating fluid as needed. In certain embodiments, fluid control valves can be solenoid-based valves. In some embodiments, the control system may include various fluid control components, such as, e.g., reservoirs, that can be used to dampen transient changes in fluid volume and/or pressure when the control system is in use.

In some embodiments, the control system may include various standard control components, such as, e.g., electronic control components or elements, indicator lights, internal or external power converters, one or more printed circuit boards, sensors, and the like. In some embodiments, the control system may include interactive features or components, such as, e.g., knobs, buttons, dials, switches and the like that can be adjusted by a user to control the cell culture system. In some embodiments, the control system may include various tubing and connection components, such as, e.g., valves, through-wall connections for tubing elements, and other interactive components.

In some embodiments, the control system may comprise a processor comprising a non-transient computer readable medium (e.g., a digital storage medium) with instructions encoded thereon (e.g., a software program) that, when executed by the processor, causes the processor to activate the control system and to operate the cell culture system in a specified manner. In certain embodiments, the control system may also include a user interface, such as, e.g., a graphical user interface.

In some embodiments, the processor may be separate from the control system and may be connected to the control system by a cable, e.g., a USB cable. In some embodiments, the processor can communicate with the control system using, e.g., a wireless network. In some embodiments the processor may be integrated with the control system such that the processor and the control system consist of a single, integrated unit.

In some embodiments, the control system can be configured to accept a user input. In some embodiments, the control system is designed to operate using existing data interface software systems, such as, e.g., Matlab™ or Labview™ in order to accept input from a user.

Control systems in accordance with embodiments of the invention may be configured to output any of a variety of commands that can be used to modulate the amount of pressure exerted by the operating fluid inside the plenum device according to a desired profile. For example, in some embodiments, the control system can output closed-loop control commands, such as waveforms, that can modulate the amplitude, duration, and frequency of cyclical pressure changes within the plenum device. In some embodiments, a control waveform can be output directly to valves containing embedded controllers, while in some embodiments a control waveform can be sent to, e.g., one or more microcontrollers that in turn control valves within the control system that can then be used to create changes in pressure within the plenum device.

In some embodiments, the control system can create signals that can be used to evoke an impulse, a square wave, a sine wave, a linear ramp, or a step response in the pressure inside the plenum device. In certain embodiments, complex parameters can be stored in the control system and used to change a waveform, and thus the pressure within the plenum device, as a function of time, such as, e.g., daily, for example, to mimic development of or changes within, e.g., a cardiovascular system. In some embodiments, the control system can be configured to output control signals to other hardware components (e.g., heaters, pumps and the like) to control other parameters that are relevant to cell culture (i.e., temperature, gas and/or fluid flow, and the like). In some embodiments, a control system can be configured to produce cyclical and/or non-cyclical pressure changes that cause the cells being cultured in the cell culture plate to experience cyclical and/or non-cyclical strains that mimic physiological conditions.

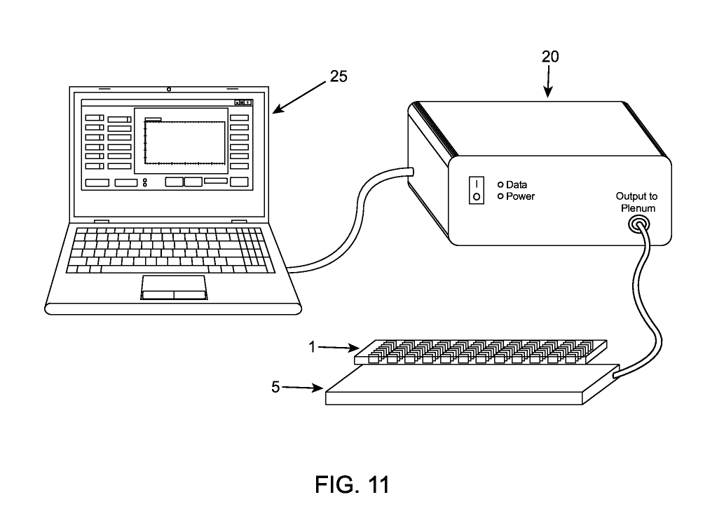

Referring now to FIG. 11, an embodiment of a cell culture system according to the present invention is depicted. In the depicted embodiment, a cell culture plate 1 is operatively coupled to a plenum device 5. The plenum device is operatively coupled to a control system 20 that can modulate the pressure inside the bounded volume of the plenum device. In the depicted embodiment, a computer processor 25 is operatively coupled to the control system.

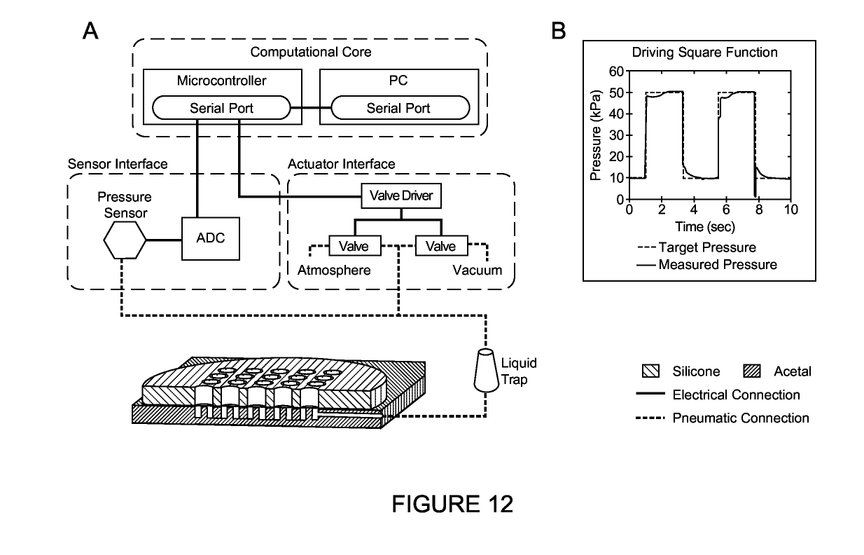

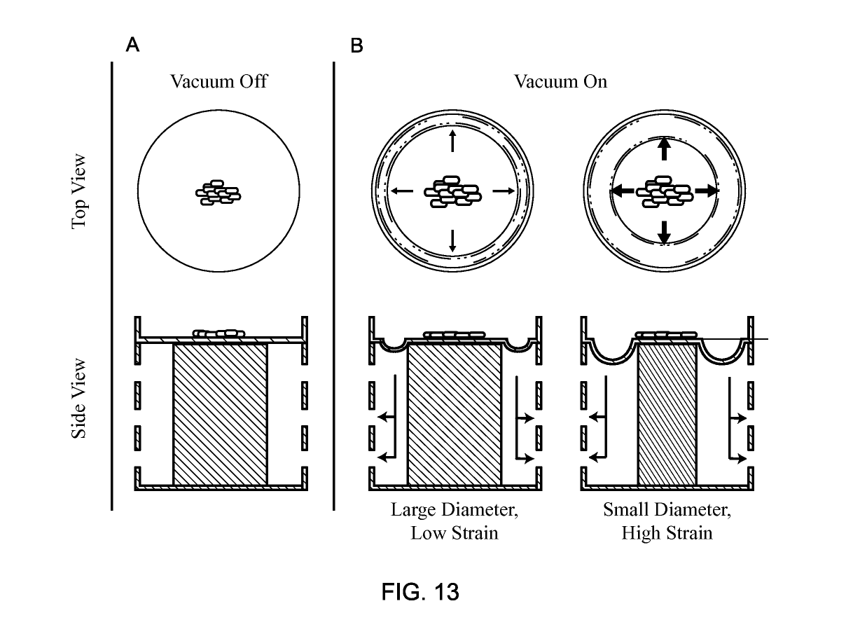

Referring now to FIG. 12, an embodiment of a control system according to the present invention is depicted. In the depicted embodiment in Panel A, a plenum device is pneumatically connected to a control system that comprises a computer processor (e.g., a PC) and a microcontroller. The microcontroller is electrically connected to a first valve that controls access to atmospheric pressure, and a second valve that controls access to a pressure source. The microcontroller is also electrically connected to an ADC that is electrically connected to a pressure sensor. The pressure sensor is pneumatically connected to the plenum device. In operation, the PC is used to send instructions to the microcontroller, and the microcontroller then modulates the pressure inside the bounded volume of the plenum device according to the instructions. Panel B of FIG. 12 depicts a waveform of pressure as a function of time that can be supplied to the plenum device by the control system. As the pressure inside the bounded volume of the plenum device is modulated by the control system, the flexible membrane material of the pliant bottoms of the cell culture wells are stretched over the strain platens of the plenum device.

### Methods

Methods in accordance with embodiments of the invention are used to apply a mechanical strain to cells that are being cultured in vitro using the cell culture systems and devices, as described above. Methods in accordance with embodiments of the invention generally involve changing the pressure, e.g., reducing the pressure, within the bounded volume of the plenum device. The reduction in pressure causes a portion of the pliant bottom of a well of the cell culture plate to stretch over a strain platen. As a result, cells that are being cultured within the well of the cell culture plate experience mechanical strain.

The cross sectional shape of a strain platen influences the type of mechanical strain that is induced when a pliant bottom of a cell culture well is stretched over a strain platen. For example, strain platens that have a circular cross-sectional shape induce biaxial/isotropic strain in the pliant bottom when the pliant bottom is stretched over the circular strain platen. Strain platens that have an oblique cross-sectional shape, in contrast, induce uniaxial/anisotropic strain in the pliant bottom when the pliant bottom is stretched over the oblique strain platen.

The cross sectional dimensions of a strain platen influence the magnitude of strain that is induced in the pliant bottom. For example, a strain platen having larger cross-sectional dimensions generally imparts a lower magnitude of strain as compared to a strain platen having smaller cross-sectional dimensions, which generally imparts a larger magnitude of strain. Accordingly, the cross-sectional dimensions of the strain platen can be adjusted to impart a desired amount of strain on the cells being cultured in the wells of the cell culture plate.

Referring now to FIG. 13, a top view and a side view of a cell culture well and a strain platen in accordance with one embodiment of the present invention are depicted. In Panel A, the vacuum source is turned off and there is no change in pressure inside the bounded volume of the plenum device. As such, the pliant bottom of the cell culture well is not stretched over the strain platen, and the cells that are being cultured on the pliant bottom are not experiencing mechanical strain. In Panel B, a top view and a side view of two different cell culture wells and two different strain platens in accordance with one embodiment of the present invention are depicted. Both of the depicted strain platens have a circular cross-sectional shape. The strain platen on the left has a larger diameter than the strain platen on the right. To carry out a method in accordance with embodiments of the present invention, a vacuum source is turned on and the pressure inside the bounded volume of the plenum device is reduced. The force created by the reduction in pressure inside the bounded volume is depicted by the arrows in the side view illustrations in Panel B. As a result of this force, the pliant bottoms of the cell culture wells are pulled downward toward the base of the plenum device and are stretched over the strain platens. The strain platen with a larger diameter induces a lower amount of strain in the pliant bottom of the cell culture well as compared to the strain platen with a smaller diameter. The cells that are being cultured on the pliant bottom of the cell culture wells experience mechanical strain, as indicated by the arrows in the top view illustrations in Panel B. The cells remain in the same focal plane while the pliant bottom of the cell culture well is stretched, which facilitates imaging of the cells.

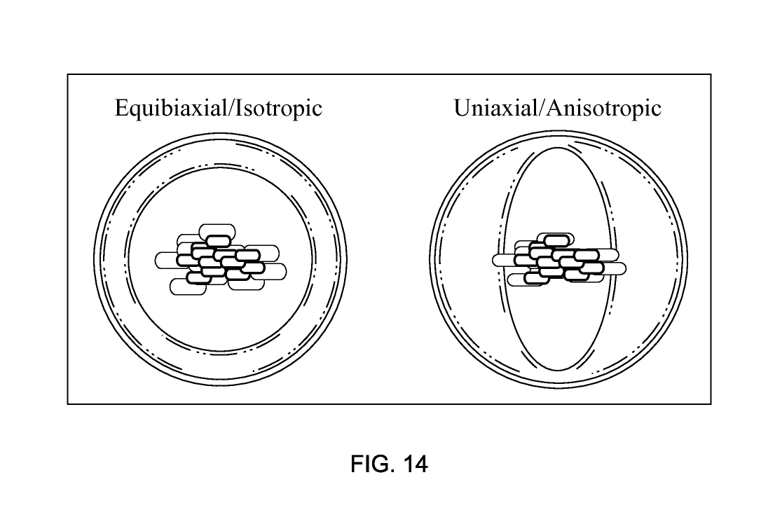

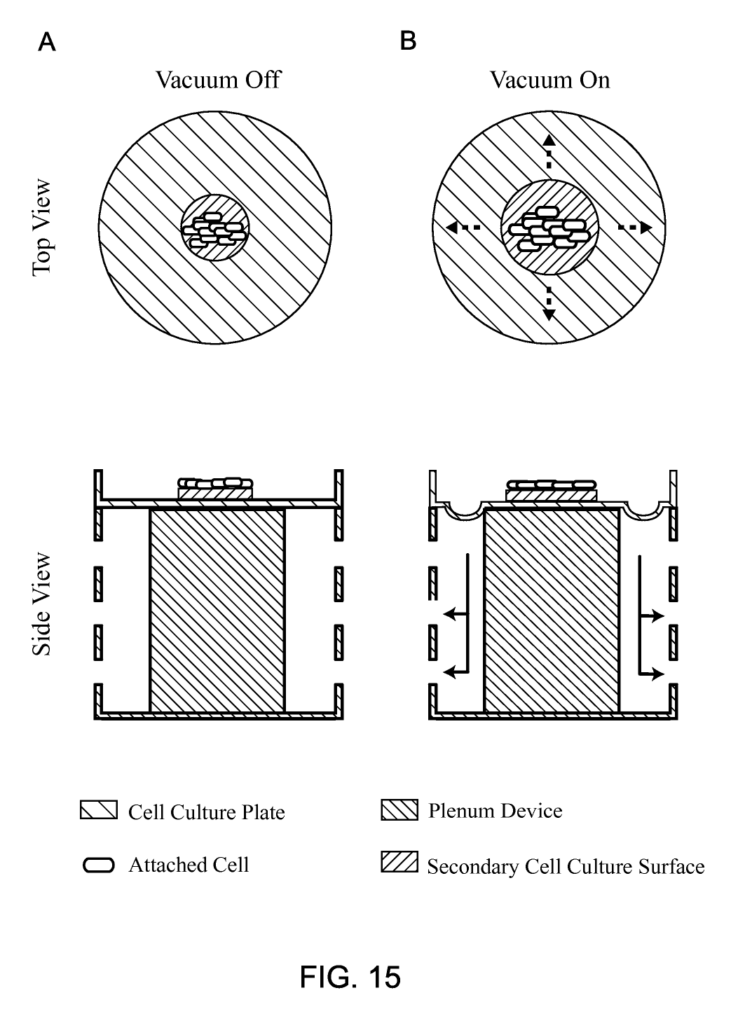

Referring now to FIG. 14, a top view illustration of two different cell culture wells according to one embodiment of the present invention is provided. Each of the depicted cell culture wells is aligned with a strain platen. In the left illustration, the cell culture well is aligned with a strain platen that has a circular cross-sectional shape. When the pliant bottom of this cell culture well is stretched over the circular strain platen, the cells that are cultured on the pliant bottom experience equibiaxial, isotropic strain. In other words, the cells experience substantially the same amount of mechanical strain in every direction. In the right illustration, the cell culture well is aligned with a strain platen that has an oval cross-sectional shape. When the pliant bottom of this cell culture well is stretched over the oval strain platen, the cells that are cultured on the pliant bottom experience uniaxial, anisotropic strain. In other words, the cells experience an amount of strain that is directionally dependent.

Referring now to FIG. 15, a top view and a side view of a cell culture well that contains a secondary cell culture surface and is aligned with a strain platen in accordance with one embodiment of the present invention are depicted. In Panel A, the vacuum source is turned off and there is no change in pressure inside the bounded volume of the plenum device. As such, the pliant bottom of the cell culture well is not stretched over the strain platen, and the secondary cell culture surface, as well as the cells that are being cultured thereon, are not experiencing mechanical strain. In Panel B, a top view and a side view of the same cell culture well, secondary cell culture surface, and strain platen are depicted. To carry out a method in accordance with embodiments of the present invention, a vacuum source is turned on and the pressure inside the bounded volume of the plenum device is reduced. The force created by the reduction in pressure inside the bounded volume is depicted by the arrows in the side view illustration in Panel B. As a result of this force, the pliant bottom of the cell culture well is pulled downward toward the base of the plenum device and is stretched over the strain platen. The secondary cell culture surface and the cells that are being cultured thereon experience mechanical strain, as indicated by the arrows in the top view illustration in Panel B. The secondary cell culture surface and the cells cultured therein remain in substantially the same focal plane while the pliant bottom of the cell culture well is stretched, which facilitates imaging of the cells.

In some embodiments, a control system, as described above, is used to control the magnitude, frequency and duration of the change in pressure in a fluid inside the bounded volume of the plenum device. For example, methods in accordance with embodiments of the invention involve operatively coupling a cell culture plate to a plenum device, and operatively coupling the plenum device to a control system that modulates the pressure within the bounded volume of the plenum device to induce mechanical strain in the pliant bottoms of the cell culture plate. In some embodiments, the control system is configured to modulate the pressure inside the bounded volume of the plenum device according to a specified pattern, such as, e.g., a specified waveform.

In some embodiments, arrays of different strain platens are used to induce different types and/or different amounts of mechanical strain in different wells of a cell culture plate. For example, methods in accordance with embodiments of the invention involve operatively coupling a cell culture plate to a plenum device, wherein the plenum device has an array of different strain platens, and operatively coupling the plenum device to a control system that modulates the pressure inside the bounded volume of the plenum device. In certain embodiments, a plenum device includes a plurality of strain platens that have the same cross-sectional shape, and two or more strain platens that have different cross-sectional dimensions. In such embodiments, a pressure change, e.g., a decrease in pressure, is applied to the bounded volume of the plenum device, and the pliant bottoms of the wells of the cell culture plate experience the same type of mechanical strain, e.g., biaxial strain, but different amounts of mechanical strain based on the cross-sectional dimensions of the strain platen that is aligned with each well.

In some embodiments, one or more of the wells of a cell culture plate are not aligned with a strain platen of the plenum device, such that the flexible membrane material of these wells is not stretched, and the cells cultured therein do not experience strain. In some embodiments, for example, a plenum device and/or a cell culture plate may be configured such that one or more wells of the cell culture plate, e.g., one or more rows, e.g., one or more columns of the cell culture plate are not aligned with strain platens such that the pliant bottoms of these wells are not stretched, and the cells cultured in these wells can serve as control samples that are not subjected to mechanical strain. Such control samples can be compared to cells that did experience mechanical strain so that the effects of the mechanical strain on the cells can be determined.

In some embodiments, the methods involve changing the magnitude, duration and/or frequency of the change in pressure inside the bounded volume of the plenum device according to a predetermined profile by using a control system. For example, in some embodiments, the control system is configured to apply a pressure waveform to the fluid within the bounded volume of the plenum device in order to apply, e.g., a cyclical pattern of pressure change to the bounded volume of the plenum device. In response, the pliant bottoms of the cell culture plate experience cyclical changes in the magnitude, frequency and duration of induced mechanical strain. The magnitude and type of induced mechanical strain is further influenced by the cross-sectional dimensions and the cross-sectional shapes, respectively, of the strain platens that are aligned with each well of the cell culture plate.

Methods in accordance with embodiments of the invention generally involve assaying the cultured cells or the cell culture medium to determine differences in, e.g., expression of specific genes, production of specific proteins, the presence of, absence of, or changes to cellular organelles and/or other cellular structures, such as, e.g., cytoskeletal proteins and the like, alignment of cells, spacing of cells, movement of cells, morphology of cells, or any other variables of interest.

Methods in accordance with embodiments of the invention may generally be used to model, or mimic a variety of conditions that may be experienced by cells in vivo. For example, methods in accordance with embodiments of the invention involve operatively coupling a cell culture plate to a plenum device and applying cyclical pressure changes to the bounded volume of the plenum device so that the pliant bottoms of the cell culture plate experience an amount of mechanical strain that is substantially the same as, or similar to the amount of mechanical strain that is experienced by cells in, e.g., tissues or organs in the body, such as, e.g., cardiac muscle. Cells can be cultured in the cell culture plate for a period of time, and the magnitude, duration and frequency of the changes in pressure can be modulated to mimic a variety of different physiological conditions, such as, e.g., elevated heart rate, elevated blood pressure, and combinations thereof.

Methods in accordance with embodiments of the invention may also involve screening candidate agents of interest, e.g., for a desired activity. For example, in some embodiments, cells are cultured in the cell culture systems described herein, and the cells are contacted with one or more candidate agents of interest to investigate the effects of the agent of interest on cells that are subjected to varying amounts of mechanical strain. In some embodiments, cells are cultured under an array of mechanical strain conditions, and the cells are contacted with a candidate agent of interest. The cells and/or the cell culture medium are assayed at various time points to evaluate the effects of the candidate agent of interest on the cells as a function of the mechanical strain that the cells experienced in culture, as well as the amount of the candidate agent that the cells were contacted with.

### Kits

Also provided are kits that at least include the subject systems and devices or components thereof, e.g., as described above, and instructions for how to use the systems and/or devices in one or more cell culture applications. In some embodiments, the kits may include a plenum device and/or a cell culture plate, such as described above.

In addition to the above components, the kits of the invention may further include instructions for practicing the subject methods. The instructions for using the systems and devices as discussed above are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub-packaging) etc. In other embodiments, the instructions are present as an electronic storage data file present on a suitable computer-readable storage medium, e.g., a digital storage medium, e.g., a CD-ROM, diskette, etc. The instructions may take any form, including complete instructions for how to use the systems and devices or as a website address with which instructions posted on the Internet may be accessed.

### EXAMPLES

### Example 1

### Finite Element Modeling of Flexible Membrane Material

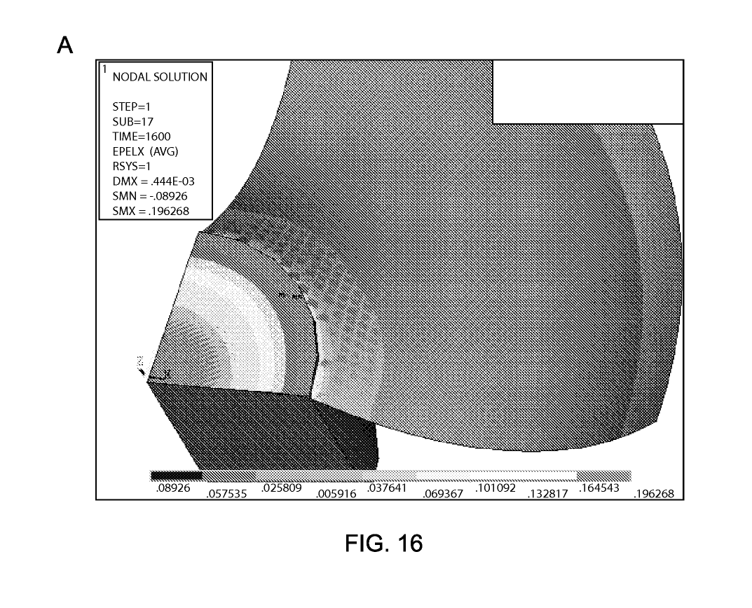

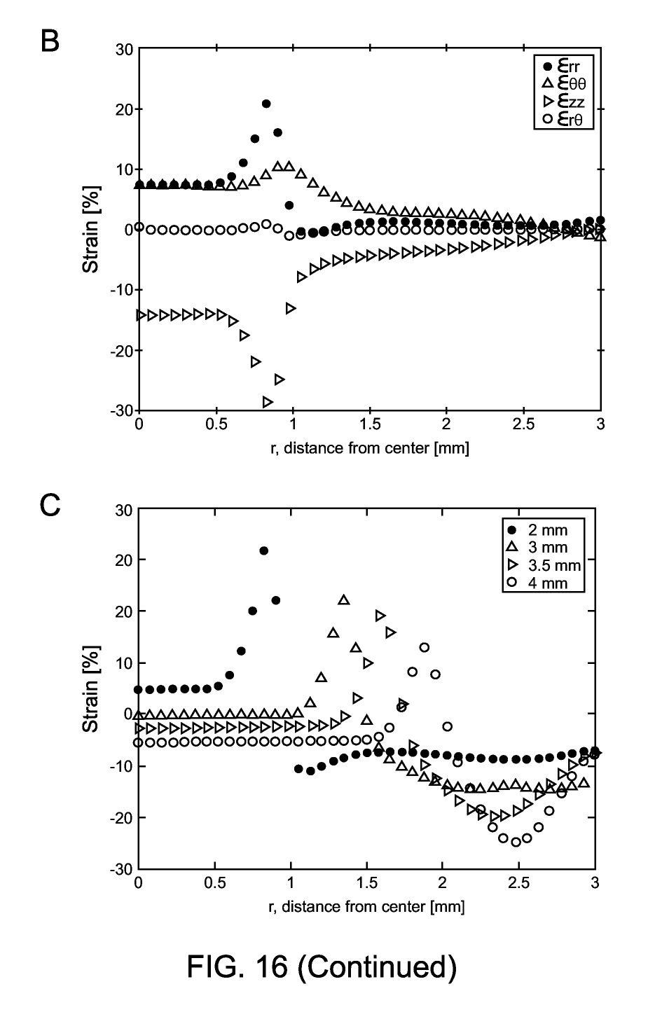

A finite element model (FEM) was used to predict the amount of strain that would be experienced by cells when the pliant bottoms of a cell culture well were stretched over a strain platen. For an applied pressure differential of 10 kPa, the model predicted that maximum strains occur in the highly stretched annular region while the region over the center of the strain platen experiences uniform strain. FIG. 16, Panel A shows an example finite element model of a 6 mm diameter cell culture well aligned with a strain platen having a circular cross-sectional shape and a diameter of 2.0 mm. Shading intensity indicates the calculated nodal strain in the radial direction for the cut-away side view. In certain areas of the model, the strain platen appears to penetrate the membrane due to the penetration tolerance of 0.1 in contact analysis. Panel B shows reported directional strains of εᵣᵣ, ε_{θθ}, ε_{rθ} and ε_{zz}, along a radial path on the x-axis on the 2 mm diameter of the strain platen. Consistent biaxial strain out to roughly 0.7 mm radius is observed. Panel C shows reported radial strain (εᵣᵣ) on the x-axis across the same radial path varying the diameter of the strain platen.

### Example 2

### Strain Calibration

In order to measure the amount of strain induced by strain platens having different cross-sectional dimensions, three different plenum devices were evaluated. The devices had circular strain platens with diameters of 4.0 mm, 3.5 mm, 3.0 mm, and 2.0 mm. The devices were operatively coupled to a cell culture plate, and a pressure difference of 10 kPa was applied. The amount of strain experienced by the flexible membrane material as it was stretched over each strain platen was evaluated.

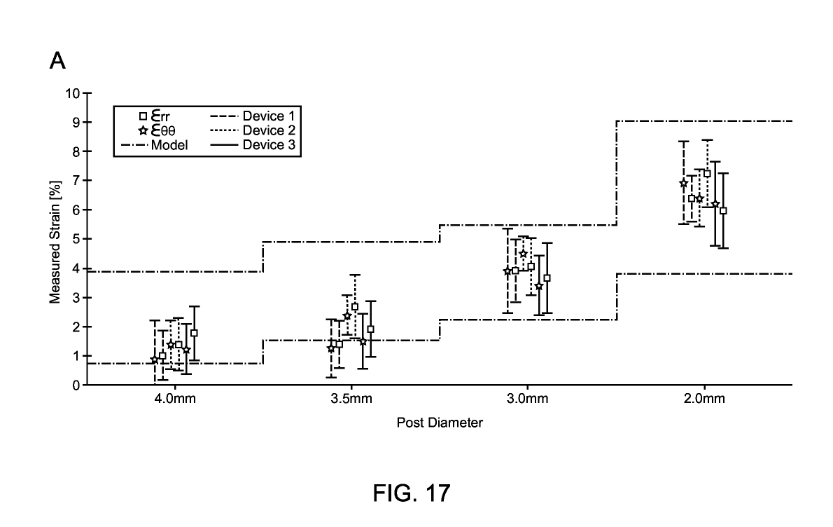

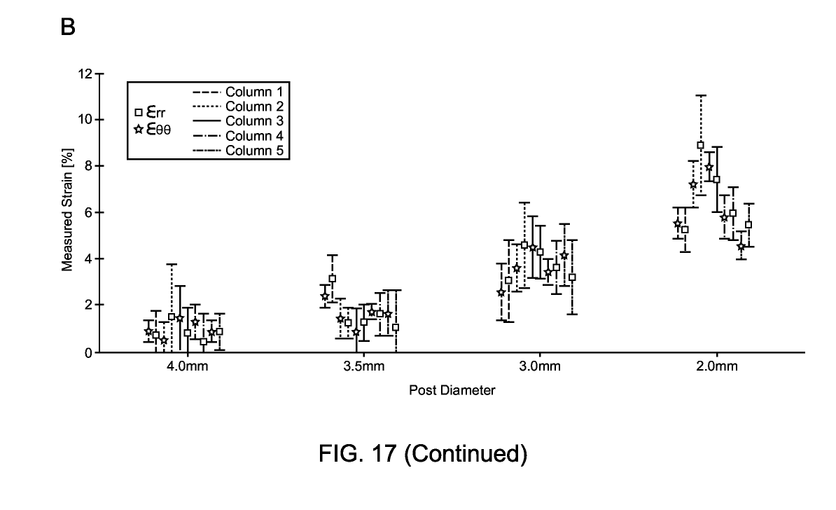

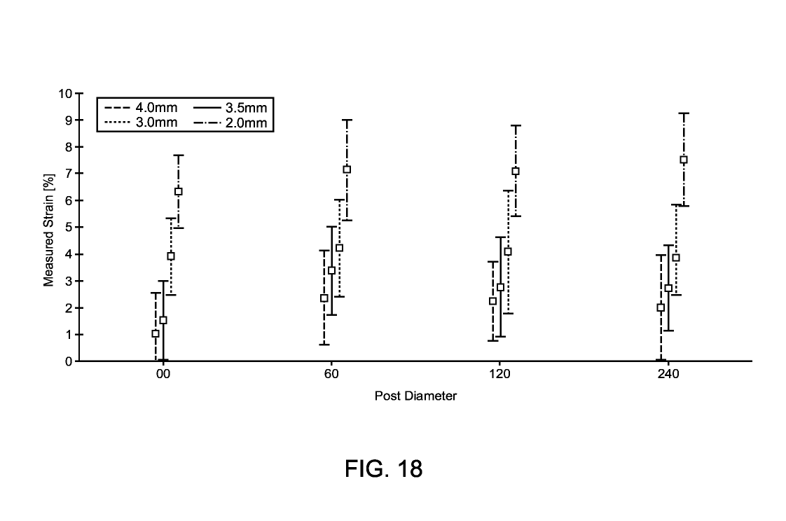

Differently sized strain platens yielded distinct strain magnitudes at 10 kPa applied pressure: 1%, 2%, 4%, and 6% for 4.0 mm, 3.5 mm, 3.0 mm, and 2.0 mm strain platens, respectively. FIG. 17, Panel A shows minimal differences among three different devices consistent with processing variability predicted by FEM. One-way ANOVA shows no significant difference among devices for the two highest strain well-sets (p>0.05). FIG. 17, Panel B shows the well-to-well variation within a single device, and a two-way ANOVA demonstrated that different strain platens create significantly different strains (p<0.001) but the well-to-well variation does not (p>0.05). As suggested by FEM, differences in strain platen diameter and spatial variations in modulus could account for the small differences seen between wells. Strain values remained consistent over 4 hours of cyclic operation after being autoclaved (FIG. 18). FIG. 17 shows mean principal strain and standard deviation as measured and computed for three separate devices (Panel A) and across a single device (Panel B) at 10 kPa applied pressure. Mean and standard deviation for a single well were determined by averaging the computed values of Lagrangian strain matrix (n=9) and the resulting values were combined (n=5) to obtain an overall device mean and standard deviation.

### Example 3

### Empirical Strain Calibration Using A Constant Strain Triangle Finite Element Model

The relationship between applied pressure and strain in the flexible membrane material was determined empirically using a two-dimensional finite element model (FEM) called a Constant Strain Triangle (CST) model. Cells were cultured in a cell culture plate that was operatively coupled to a plenum device. A vacuum pressure ranging from 10-50 kPa was applied to the device in increments of 10 kPa. At each vacuum level (including zero vacuum), a photograph was taken. This process was repeated for each of three different strain platen diameters. The result of this process was a set of photographs of each cell culture at increasing levels of strain.

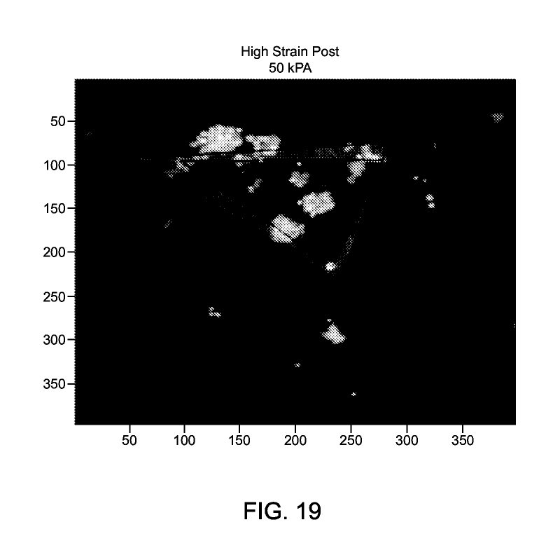

The resulting set of photographs was then used to calculate the strain experienced by the cells using CST FEM methodology. For each set of photographs, three cell nuclei were chosen to serve as the three nodes of a CST. A custom MATLAB script was used to track the movement of these nodes at each pressure level, and using this information, the strain was calculated for each pressure level and strain platen size. FIG. 19 shows an example of the nucleus tracking results. The two triangles shown in the figure are superimposed over the photograph of the cell culture when it is not under pressure. The smaller triangle is the original triangle, and the larger triangle is the size of the triangle (as defined by the location of the tracked nuclei) when a pressure of 50 kPa was applied to the membrane. The results show that the cells experienced mechanical strain, as expected, when the vacuum pressure source was applied to the system.

### Example 4

### Cultures of Strained Cells

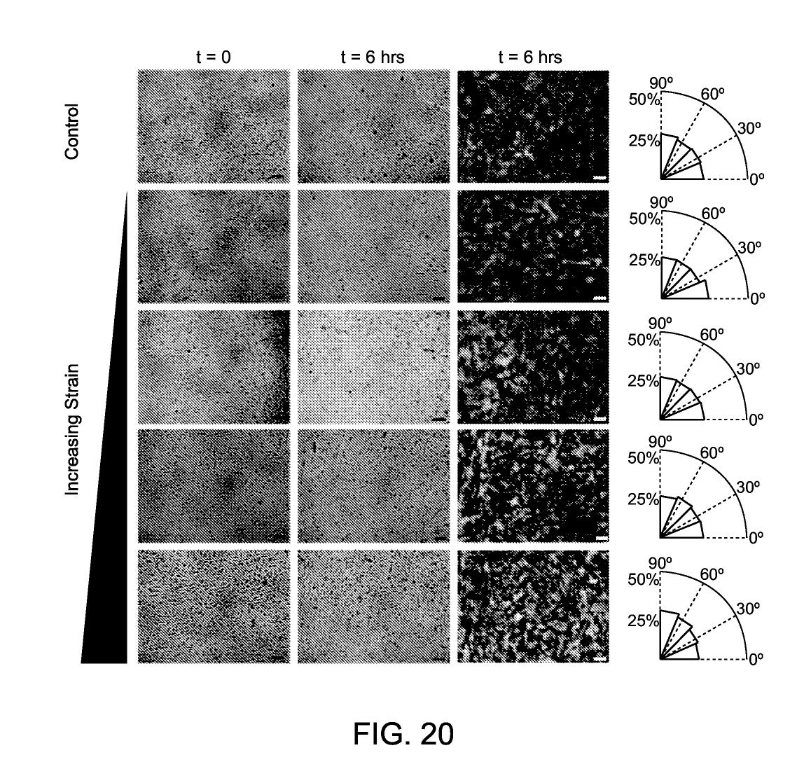

Cells were cultured in cell culture plates and were subjected to different amounts of mechanical strain. Specifically, C2C12 skeletal myoblasts were used. The cells adhered to and spread uniformly across all wells of the cell culture plate after 12 hours of culture, demonstrating cell compatibility. In several reports, isolated C2C12 and other cells have been observed to align perpendicular to cyclic uniaxial strains above 1 Hz, thereby minimizing the strain along the direction of stress fibers (actin filaments). Under biaxial strain conditions, the nuclei of C2C12 cells realigned after cyclic loading at higher strains while they remained randomly oriented with low or no applied strain (FIG. 20). At higher strains, cells and their nuclei realigned circumferentially, i.e., perpendicular (90°) to radial vectors as calculated by custom MATLAB™ code (last column of FIG. 20). Circumferential alignment was greatest at the outer perimeter and was consistent with realignment behavior under uniaxial cyclic loading.

Bright field and fluorescent images at t=0 and t=6 hrs after application of cyclic strain at 10 kPa magnitude and 1 Hz frequency are shown in FIG. 20. Scale bar=100 μm. Nuclei and F-actin within the cells were stained with DAPI and rhodamine-phalloidin, and nucleus alignment was characterized by custom MATLAB™ code. Mean angular distribution of nucleus orientation (absolute angle between long axis of nucleus and radial vector to center of well) is depicted in the adjacent radial histogram (n=5).

Notwithstanding the appended claims, the disclosure is also defined by the following clauses:

1. A plenum device, the plenum device comprising: a base component; a wall component configured to define a bounded volume having a bottom that is a surface of the base component; and a pressure modulator configured to provide a substantially uniform pressure inside the bounded volume upon application of an external pressure source via an internal side opening in the wall component.

2. The plenum device according to Clause 1, wherein the pressure modulator comprises one or more structures extending from at least one of the surface of the base component and an inner surface of the wall component.

3. The plenum device according to Clause 2, wherein the pressure modulator comprises a plurality of strain platens extending from at least one of the surface of the base component and an inner surface of the wall component.

4. The plenum device according to Clause 2, wherein the structures are uniformly spaced.

5. The plenum device according to Clause 2, wherein the structures are non-uniformly spaced.

6. The plenum device according to Clause 1, wherein the device is configured to impart a mechanical strain on a flexible membrane material that forms a pliant bottom of a well of a cell culture plate operatively coupled thereto.

7. The plenum device according to Clause 6, wherein the strain is a substantially isotropic mechanical strain.

8. The plenum device according to Clause 6, wherein the strain is a substantially anisotropic mechanical strain.

9. The plenum device according to Clause 6, wherein the device is configured to impart a mechanical strain gradient on two or more wells of a cell culture plate operatively coupled thereto.

10. The plenum device according to Clause 9, wherein the device comprises two or more strain platens configured to impart different mechanical strains on different wells of a cell culture plate operatively coupled thereto.

11. The plenum device according to Clause 10, wherein the two or more strain platens have different cross-sectional shapes.

12. The plenum device according to Clause 10, wherein the two or more strain platens have different cross-sectional dimensions.

13. The plenum device according to Clause 1, wherein the base component and the wall component are integrated into a single unit.

14. The plenum device according to Clause 1, wherein the base component and the wall component are separable from one another.

15. The plenum device according to Clause 1, wherein the wall component comprises a single internal side opening.

16. The plenum device according to Clause 1, wherein the wall component comprises two or more internal side openings.

17. The plenum device according to Clause 1, wherein the bounded volume has a volume ranging from about 10 to about 120 cubic centimeters.

18. A cell culture system comprising: (a) a plenum device comprising: (i) a base component; (ii) a wall component configured to define a bounded volume having a bottom that is a surface of the base component; and (iii) a pressure modulator configured to provide a substantially uniform pressure in the bounded volume upon application of an external pressure source via an internal side opening in the wall component; and (b) a cell culture plate comprising two or more cell culture wells, each well having a pliant bottom.

19. The system according to Clause 18, wherein the pressure modulator comprises one or more structures extending from at least one of the surface of the base component and an inner surface of the wall component.

20. The system according to Clause 19, wherein the pressure modulator comprises a plurality of strain platens extending from at least one of the surface of the base and an inner surface of the wall component.

21. The system according to Clause 19, wherein the structures are uniformly spaced.

22. The system according to Clause 19, wherein the structures are non-uniformly spaced.

23. The system according to Clause 18, wherein the device is configured to impart a mechanical strain on the pliant bottoms of the wells of the cell culture plate.

24. The system according to Clause 23, wherein the strain is a substantially isotropic mechanical strain.

25. The system according to Clause 23, wherein the strain is a substantially anisotropic mechanical strain.

26. The system according to Clause 23, wherein the plenum device is configured to impart a mechanical strain gradient on the pliant bottoms of two or more wells of the cell culture plate.

27. The system according to Clause 26, wherein the device comprises two or more strain platens configured to impart different mechanical strains on the pliant bottoms of two or more wells of the cell culture plate.

28. The system according to Clause 27, wherein the two or more strain platens have different cross-sectional shapes.

29. The system according to Clause 27, wherein the two or more strain platens have different cross-sectional dimensions.

30. The system according to Clause 18, wherein the plenum device is configured to maintain at least a portion of the pliant bottoms of the wells of the cell culture plate in substantially the same focal plane when a mechanical strain is imparted to the pliant bottoms.

31. The system according to Clause 18, further comprising a control system configured to modulate the pressure inside the bounded volume of the plenum device.

32. The system according to Clause 29, wherein the control system is a pneumatic control device.

33. The system according to Clause 29, wherein the control system is a hydraulic control device.

34. The system according to Clause 29, wherein the control system is a closed-loop control system.

35. The system according to Clause 29, wherein the control system is configured to modulate the pressure in the bounded volume of the plenum device according to a waveform.

36. The system according to Clause 29, wherein the control system comprises a microprocessor.

37. The system according to Clause 34, wherein the microprocessor comprises a program that, when executed, causes the control system to modulate the pressure in the bounded volume of the plenum device.

38. The system according to Clause 35, wherein the program is configured to accept a user input.

39. The system according to Clause 36, wherein the program is configured to display a graphical user interface.

40. The system according to Clause 18, further comprising a fluid transport system operatively coupled to the cell culture plate.

41. The system according to Clause 40, wherein the fluid transport system is configured to deliver one or more fluids to one or more wells of the cell culture plate.

42. The system according to Clause 40, wherein the fluid transport system is configured to withdraw a quantity of fluid from one or more wells of the cell culture plate.

43. The system according to Clause 40, wherein the fluid transport system comprises a fluid reservoir.

44. The system according to Clause 18, wherein the cell culture plate is operatively coupled to a cell culture incubator.

45. The system according to Clause 44, wherein the incubator is configured to modulate and/or control at least one of the temperature and the gaseous environment of the wells of the cell culture plate.

46. The system according to Clause 18, further comprising a pressure source.

47. The system according to Clause 18, further comprising an imaging device.

48. The system according to Clause 18, wherein the cell culture plate comprises a lid that is configured to allow retrieval of the contents of one or more wells of the cell culture plate.

49. The system according to Clause 18, further comprising a stimulation device configured to deliver an electrical stimulation to the contents of one or more wells of the cell culture plate.

50. The system according to Clause 49, wherein the stimulation device is operatively coupled to a well of the cell culture plate.

51. The system according to Clause 49, wherein the cell culture plate comprises an electrode array.

52. The system according to Clause 49, wherein the electrode array comprises electrodes operatively coupled to two or more wells of the cell culture plate.

53. The system according to Clause 18, wherein the cell culture plate comprises a composite structure that is configured to mechanically stabilize the cell culture plate.

54. The system according to Clause 18, wherein the cell culture plate is configured to promote protein attachment to the wells of the cell culture plate.

55. The system according to Clause 18, wherein the cell culture plate is configured to promote binding of a non-biological material to the cell culture substrate of the cell culture plate.

56. The system according to Clause 18, wherein the cell culture plate further comprises a secondary cell culture surface.

57. A cell culture method, the method comprising: (a) placing a cell in a cell culture plate of a cell culture system comprising: (i) a plenum device comprising: a base component; a wall component configured to define a bounded volume having a bottom that is a surface of the base component; and a pressure modulator configured to provide a substantially uniform pressure in the bounded volume upon application of an external pressure source via an internal side opening in the wall component; and (ii) a cell culture plate comprising two or more cell culture wells, each having a pliant bottom; and (b) applying a pressure to the bounded volume of the plenum device to impart a mechanical strain to the pliant bottoms of the wells of the cell culture plate, thereby imparting a mechanical strain to the cell.

58. The method according to Clause 57, wherein the method comprises imparting a substantially isotropic mechanical strain to the pliant bottoms.

59. The method according to Clause 57, wherein the method comprises imparting a substantially anisotropic mechanical strain to the pliant bottoms.

60. The method according to Clause 57, wherein the plenum device is configured to impart a mechanical strain gradient to the pliant bottoms of the cell culture plate.

61. The method according to Clause 57, wherein the cell is a stem cell.

62. The method according to Clause 57, wherein the cell is attached to a tissue culture scaffold.

63. A method of evaluating the activity of a candidate agent, the method comprising: (a) contacting a cell with a candidate agent, wherein the cell is present in a cell culture system comprising: (i) a plenum device comprising: a base component; a wall component configured to define a bounded volume having a bottom that is a surface of the base component; and a pressure modulator configured to provide a substantially uniform pressure in the bounded volume upon application of an external pressure source via an internal side opening in the wall component; and (ii) a cell culture plate comprising two or more cell culture wells each having a pliant bottom; (b) modulating the pressure in the bounded volume of the plenum device to induce a mechanical strain in the pliant bottoms, thereby imparting a mechanical strain to the cell; and (c) assaying the cell and/or the cell culture medium to evaluate the activity of the candidate agent.

64. The method according to Clause 63, wherein the method comprises imparting a substantially isotropic mechanical strain to the pliant bottoms.

65. The method according to Clause 63, wherein the method comprises imparting a substantially anisotropic mechanical strain to the pliant bottoms.

66. The method according to Clause 63, wherein the method comprises imparting a mechanical strain gradient to the pliant bottoms.

67. The method according to Clause 63, wherein the cell is attached to a tissue culture scaffold.

68. The method according to Clause 63, wherein the cell is a stem cell.

69. The method according to Clause 68, wherein the candidate agent is evaluated for cellular differentiation activity.

70. The method according to Clause 63, wherein the candidate agent is evaluated for gene expression modulatory activity.

71. The method according to Clause 63, wherein the candidate agent is evaluated for protein production modulatory activity.

72. The method according to Clause 63, wherein the candidate agent is evaluated for signaling pathway modulatory activity.

73. The method according to Clause 63, wherein the method is a high throughput method.

74. A kit comprising: (a) a plenum device comprising: a base component; a wall component configured to define a bounded volume having a bottom that is a surface of the base component; and a pressure modulator configured to provide a substantially uniform pressure in the bounded volume upon application of an external pressure source via an internal side opening in the wall component; and (b) a cell culture plate comprising two or more cell culture wells, each having a pliant bottom.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A plenum device, the plenum device comprising:
a base component;
a wall component configured to define a bounded volume having a bottom that is a surface of the base component; and
a pressure modulator configured to provide a substantially uniform pressure inside the bounded volume upon application of an external pressure source via an internal side opening in the wall component.

2. The plenum device according to claim 1, wherein the pressure modulator comprises one or more structures extending from at least one of the surface of the base component and an inner surface of the wall component.

3. The plenum device according to claim 1, wherein the pressure modulator comprises a plurality of strain platens extending from at least one of the surface of the base component and an inner surface of the wall component.

4. The plenum device according to claim 1, wherein the device is configured to impart a mechanical strain on a flexible membrane material that forms a pliant bottom of a well of a cell culture plate operatively coupled thereto.

5. (canceled)

6. The plenum device according to claim 1, wherein the device comprises two or more strain platens configured to impart different mechanical strains on different wells of a cell culture plate operatively coupled thereto.

7. (canceled)

8. The plenum device according to claim 1, wherein the two or more strain platens have different cross-sectional dimensions.

9. The plenum device according to claim 1, wherein the wall component comprises a single internal side opening.

10. A cell culture system comprising:
(a) a plenum device comprising:
(i) a base component;
(ii) a wall component configured to define a bounded volume having a bottom that is a surface of the base component; and
(iii) a pressure modulator configured to provide a substantially uniform pressure in the bounded volume upon application of an external pressure source via an internal side opening in the wall component; and
(b) a cell culture plate comprising two or more cell culture wells, each well having a pliant bottom.

11. The system according to claim 10, wherein the pressure modulator comprises one or more structures extending from at least one of the surface of the base component and an inner surface of the wall component.

12. The system according to claim 10, wherein the pressure modulator comprises a plurality of strain platens extending from at least one of the surface of the base and an inner surface of the wall component.

13. The system according to claim 10, wherein the plenum device is configured to impart a mechanical strain on a pliant bottom of a well of the cell culture plate.

14. The system according to claim 10, wherein the plenum device comprises two or more strain platens configured to impart different mechanical strains on the pliant bottoms of two or more wells of the cell culture plate.

15. The system according to claim 10, wherein the plenum device is configured to maintain at least a portion of the pliant bottoms of the wells of the cell culture plate in substantially the same focal plane when a mechanical strain is imparted to the pliant bottoms.

16-18. (canceled)

19. The system according to claim 10, further comprising a fluid transport system operatively coupled to the cell culture plate.

20. The system according to claim 10, further comprising a stimulation device configured to deliver an electrical stimulation to the contents of one or more wells of the cell culture plate.

21. The system according to claim 10, wherein the cell culture plate is configured to promote protein or cell attachment to the wells of the cell culture plate.

22. The system according to claim 10, wherein the cell culture plate further comprises a secondary cell culture surface.

23. The system according to claim 10, further comprising a control system configured to modulate the pressure inside the bounded volume of the plenum device.

24. The system according to claim 23, wherein the control system comprises a microprocessor.

25. The system according to claim 10, further comprising an imaging device.
